(19) United States
(12) Patent Application Publication
(10) Pub. No.: US 2014/0031525 A1
(43) Pub. Date: Jan. 30, 2014

(54) MUTANT G-PROTEIN COUPLED RECEPTOR PROTEINS AND METHODS FOR PRODUCING THEM

(71) Applicants: Nathan Jacob Robertson, Hertfordshire (GB); Fiona Hamilton Marshall, Hertfordshire (GB)

(72) Inventors: Nathan Jacob Robertson, Hertfordshire (GB); Fiona Hamilton Marshall, Hertfordshire (GB)

(73) Assignee: Heptares Therapeutics Limited, Hertfordshire (GB)

(21) Appl. No.: 13/980,497

(22) PCT Filed: Jan. 20, 2012
(86) PCT No.: PCT/GB12/50129

§ 371 Date: Oct. 15, 2013

Related U.S. Application Data
(60) Provisional application No. 61/435,099, filed on Jan. 21, 2011.

(51) Int. Cl. C07K 14/47

(52) U.S. Cl. CPC C07K 14/4722
USPC 530/350

(57) ABSTRACT

A method for producing a mutant G-protein coupled receptor (GPCR) with increased stability relative to a parent GPCR, the method comprising making one or more mutations in the amino acid sequence that defines a Class 1 parent GPCR, wherein (i) the one or more mutations are located within a window of i plus or minus 5 residues, where i is the position of amino acid residue 3.55 in the parent GPCR, and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, to provide one or more mutants of the parent GPCR with increased stability.

The present invention relates to mutant 7-transmembrane spanning receptors (7-TMRs) or G protein coupled receptors (GPCRs) and methods for selecting those with increased stability. In particular, it relates to the selection and preparation of mutant GPCRs which have increased stability under a particular condition compared to their respective parent proteins. Such proteins are more likely to be crystallisable, and hence amenable to structure determination, than the parent proteins. They are also useful for drug discovery and development studies.

Over the past 20 years the rate of determination of membrane protein structures has gradually increased, but most success has been in crystallising membrane proteins from bacteria rather than from eukaryotes [1]. Bacterial membrane proteins have been easier to overexpress using standard techniques in Escherichia coli than eukaryotic membrane proteins [2,3] and the bacterial proteins are sometimes far more stable in detergent, detergent-stability being an essential prerequisite to purification and crystallisation. Genome sequencing projects have also allowed the cloning and expression of many homologues of a specific transporter or ion channel, which also greatly improves the chances of success during crystallisation. Although the structures of over 100 unique polytopic integral membrane proteins have been determined (see http://blanco.biomol.uci.edu/), less than 10% of these membrane proteins are of mammalian origin and over half were purified from natural sources and are stable in detergent solutions. Apart from the difficulties in overexpressing eukaryotic membrane proteins, they often have poor stability in detergent solutions, which severely restricts the range of crystallisation conditions that can be explored without their immediate denaturation or precipitation. Ideally, membrane proteins should be stable for many days in any given detergent solution, but the detergents that are best suited to growing diffraction-quality crystals tend to be the most destabilising detergents ie those with short aliphatic chains and small or charged head groups. It is also the structures of human membrane proteins that we would like to solve, because these are required to help the development of therapeutic agents by the pharmaceutical industry; often there are substantial differences in the pharmacology of receptors, channels and transporters from different mammals, whilst yeast and bacterial genomes may not include any homologous proteins. There is thus an overwhelming need to develop a generic strategy that will allow the production of detergent-stable eukaryotic integral membrane proteins for crystallisation and structure determination and potentially for other purposes such as drug screening, bioassay and biosensor applications.

Membrane proteins have evolved to be sufficiently stable in the membrane to ensure cell viability, but they have not evolved to be stable in detergent solution, suggesting that membrane proteins could be artificially evolved and detergent-stable mutants isolated [4]. This was subsequently demonstrated for two bacterial proteins, diacylglycerol kinase (DGK) [5,6] and bacteriorhodopsin [7]. Random mutagenesis of DGK identified specific point mutations that increased thermostability and, when combined, the effect was additive so that the optimally stable mutant had a half-life of 35 minutes at 80° C. compared with a half-life of 6 minutes at 55° C. for the native protein [6]. It was shown that the trimer of the detergent-resistant DGK mutant had become stable in SDS and it is thus likely that stabilisation of the oligomeric state played a significant role in thermostabilisation. Although the aim of the mutagenesis was to produce a membrane protein suitable for crystallisation, the structure of DGK has yet to be determined and there have been no reports of successful crystallization. A further study on bacteriorhodopsin by cysteine-scanning mutagenesis along helix B demonstrated that it was not possible to predict which amino acid residues would lead to thermostability upon mutation nor, when studied in the context of the structure, was it clear why thermostabilisation had occurred [7].

GPCRs constitute a very large family of proteins that control many physiological processes and are the targets of many effective drugs. Thus, they are of considerable pharmacological importance. A list of GPCRs is given in Foord et al (2005) Pharmacol Rev. 57, 279-288, which is incorporated herein by reference. GPCRs are generally unstable when isolated, and until recently, it has not been possible to crystallise any except bovine rhodopsin, which is exceptionally stable in its native unilluminated state.

By GPCRs we include all 7-TMRs within the GPCR superfamily, including receptors that signal to G proteins as well as those receptors which do not signal to G proteins.

GPCRs are druggable targets and reference is made to Overington et al (2006) Nature Rev. Drug Discovery 5, 993-996 which indicates that more than a quarter of current drugs target GPCRs. There are 52 GPCR targets for orally available drugs out of a total of 186 total targets in this category. GPCRs are thought to exist in multiple distinct conformations which are associated with different pharmacological classes of ligand such as agonists and antagonists, and to cycle between these conformations in order to function (Kenakin T. (1997) Ann N Y Acad Sci 812, 116-125).

The inventors have previously developed various methodologies for selecting mutations that improve the stability of GPCRs, and, in addition, that preferentially lock the receptor in a specific biologically relevant conformation. Such methods are described in WO 2008/114020 and in WO 2009/071914, incorporated herein by reference.

The inventors have now developed a further method for producing mutant GPCRs with increased stability relative to a parent GPCR. Specifically, they have identified that mutating any of amino acid residues 3.55, 5.63 and 7.42 (as defined by the Ballesteros numbering system described below) can be used to provide mutant GPCRs with increased stability in a particular conformation.

Accordingly, a first aspect of the invention provides a method for producing a mutant G-protein coupled receptor (GPCR) with increased stability relative to a parent GPCR, the method comprising making one or more mutations in the amino acid sequence that defines a Class 1 parent GPCR, wherein (i) the one or more mutations are located within a window of i plus or minus 5 residues, where i is the position of amino acid residue 3.55 in the parent GPCR and/or (ii) the one or more mutations are located within a window of i minus 2 to i residues, where i is the position of amino acid residue 5.63 in the parent GPCR and/or (iii) the one or more mutations are located within a window of i minus 4 to i plus 1 residues, where i is the position of amino acid residue 7.42 in the parent GPCR, to provide one or more mutants of the parent GPCR with increased stability.

Suitable GPCRs for use in the practice of the invention include, but are not limited to adenosine receptor, in particular adenosine A_{2A} receptor (gene name: ADORA2A), muscarinic receptor, serotonin receptor (eg 5HT_{2C}; gene name HTR2C), β-adrenergic receptor (e.g. βAR-1; gene name: ADRB1), neurotensin receptor (NTS₁; gene name: NTSR1), and orexin receptor (e.g. OX₂; gene name: HTR2C). In addition, the International Union of Pharmacology produces a list of GPCRs (Foord et al (2005) Pharmacol. Rev. 57, 279-288, incorporated herein by reference and this list is periodically updated at http://www.iuphar-db.org/GPCR/ReceptorFamiliesForward). It will be noted that GPCRs are divided into different classes, principally based on their amino acid sequence similarities, for example Classes 1, 2 and 3 whose archetypes are rhodopsin, the secretin receptor and the metabotropic glutamate receptor 1. GPCRs are also divided into families by reference to the natural ligands to which they bind. All Class 1 GPCRs, including 7-TMRs in the superfamily of GPCRs, are included in the scope of the invention. Thus, the GPCR may be any of a mutant adenosine receptor, a mutant (3-adrenergic receptor, a mutant neurotensin receptor, a mutant muscarinic acid receptor, a mutant 5-hydroxytryptamine receptor, a mutant adrenoceptor, a mutant anaphylatoxin receptor, a mutant angiotensin receptor, a mutant apelin receptor, a mutant bombesin receptor, a mutant bradykinin receptor, a mutant cannabinoid receptor, a mutant chemokine receptor, a mutant cholecystokinin receptor, a mutant dopamine receptor, a mutant endothelin receptor a mutant free fatty acid receptor, a mutant bile acid receptor, a mutant galanin receptor, a mutant motilin receptor, a mutant ghrelin receptor, a mutant glycoprotein hormone receptor, a mutant GnRH receptor, a mutant histamine receptor, a mutant KiSS1-derived peptide receptor, a mutant leukotriene and lipoxin receptor, a mutant lysophospholipid receptor, a mutant melanin-concentrating hormone receptor, a mutant melanocortin receptor, a mutant melatonin receptor, a mutant neuromedin U receptor, a mutant neuropeptide receptor, a mutant N-formylpeptide family receptor, a mutant nicotinic acid receptor, a mutant opiod receptor, a mutant opsin-like receptor, a mutant orexin receptor, a mutant P2Y receptor, a mutant peptide P518 receptor, a mutant platelet-activating factor receptor, a mutant prokineticin receptor, a mutant prolactin-releasing peptide receptor, a mutant prostanoid receptor, a mutant protease-activated receptor, a mutant relaxin receptor, a mutant somatostatin receptor, a mutant SPC/LPC receptor, a mutant tachykinin receptor, a mutant trace amino receptor, a mutant thryotropin-releasing hormone receptor, a mutant urotensin receptor, a mutant vasopressin/oxytocin receptor, a mutant orphan GPCR, a mutant calcitonin receptor, a mutant corticotropin releasing factor receptor, a mutant glucagon receptor, a mutant parathyroid receptor, a mutant VIP/PACAP receptor, a mutant LNB7TM receptor, a mutant GABA receptor, a mutant metabotropic glutamate receptor, and a mutant calcium sensor receptor (see Table 1 of Foord et al (2005) Pharmacol. Rev. 57, 279-288, incorporated herein by reference).

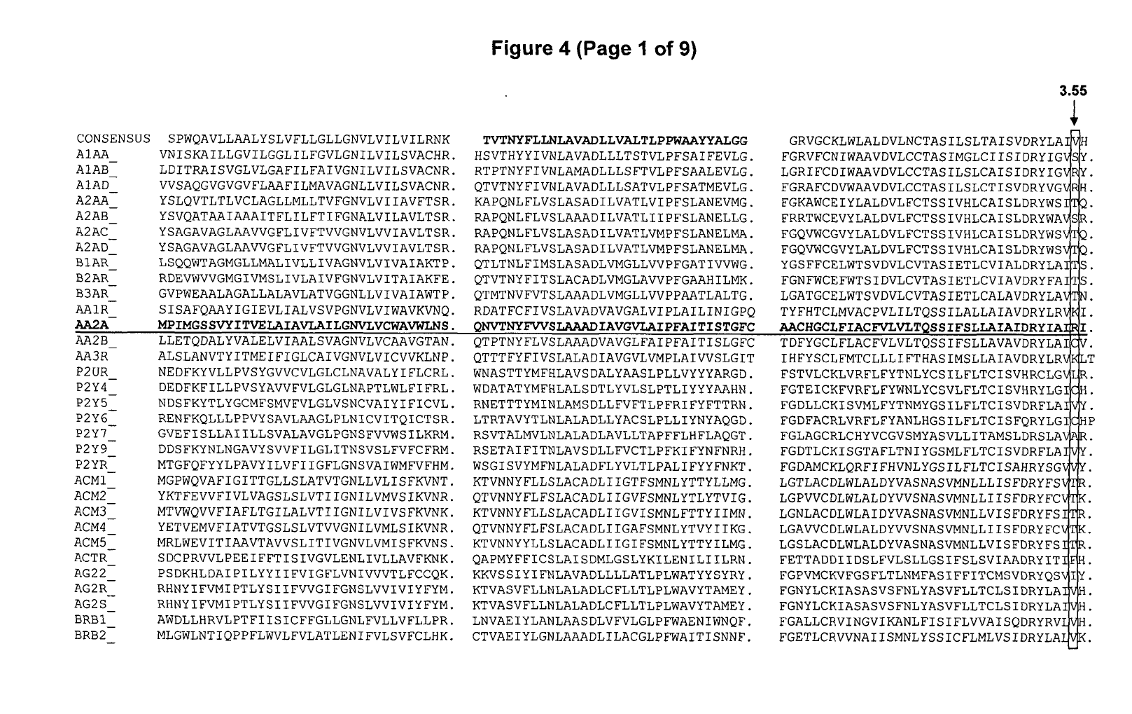

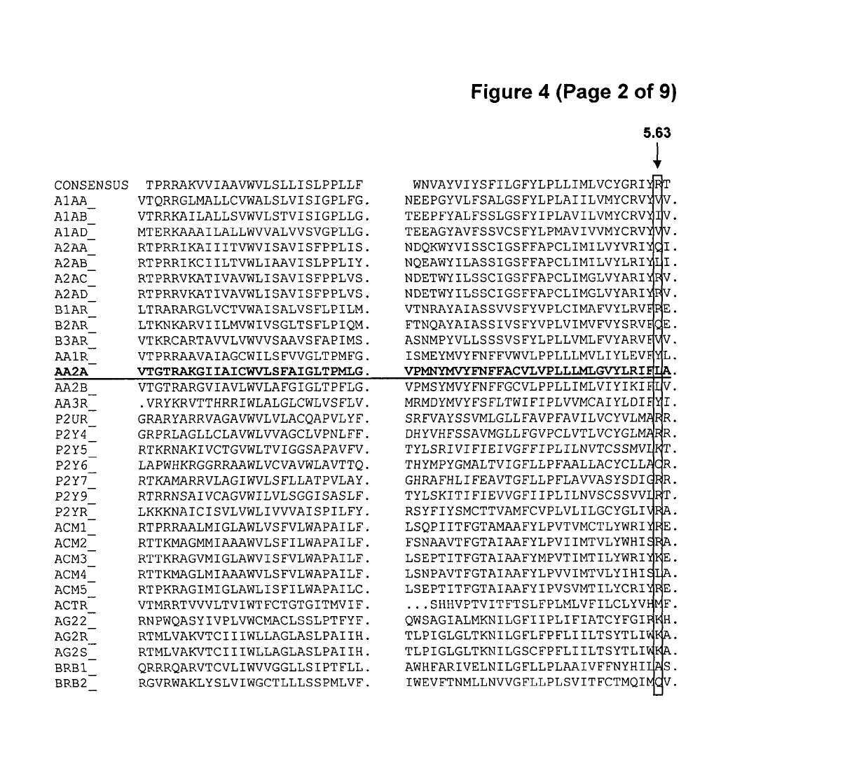

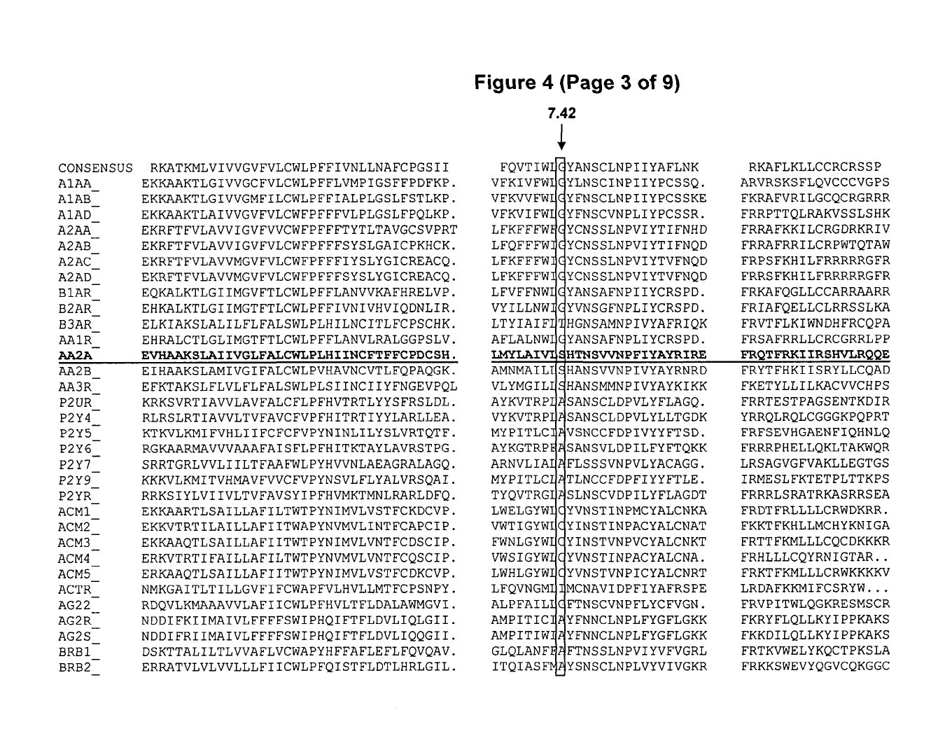

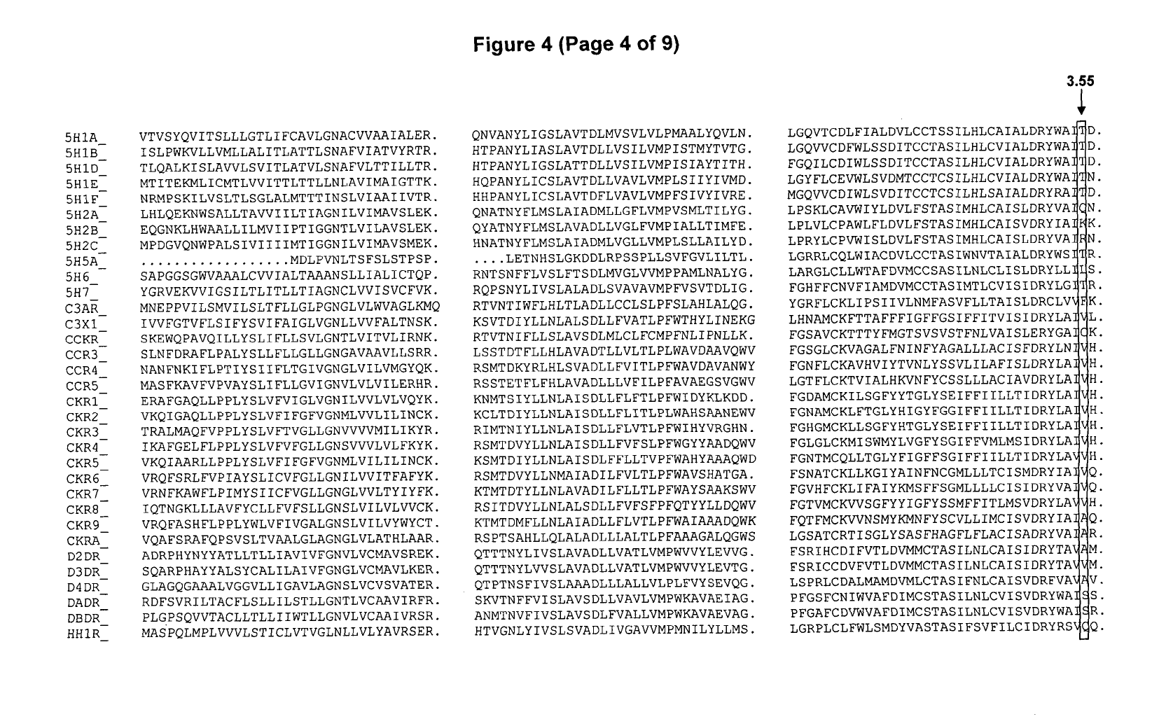

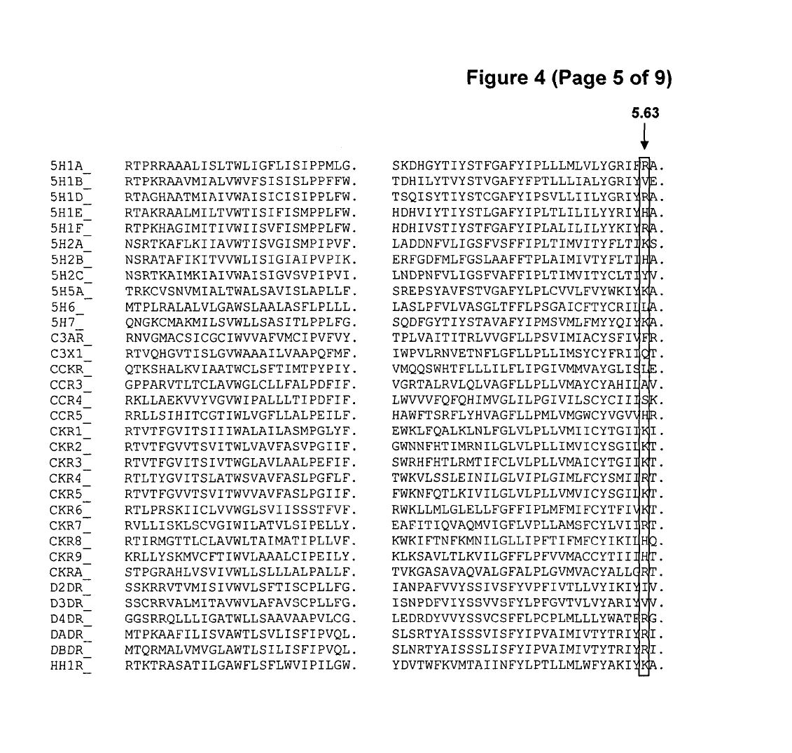

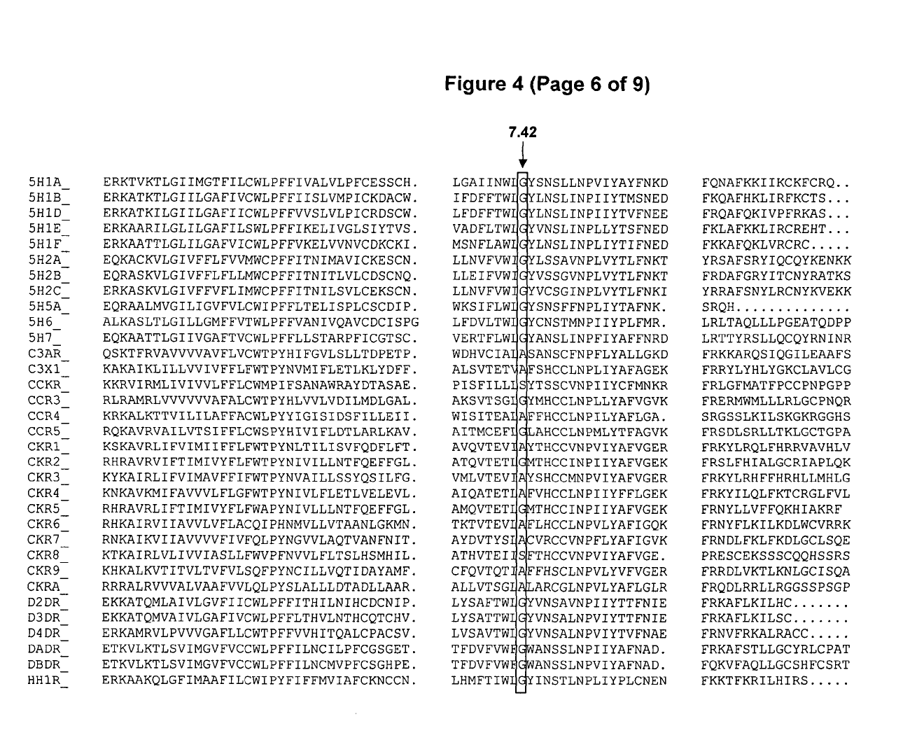

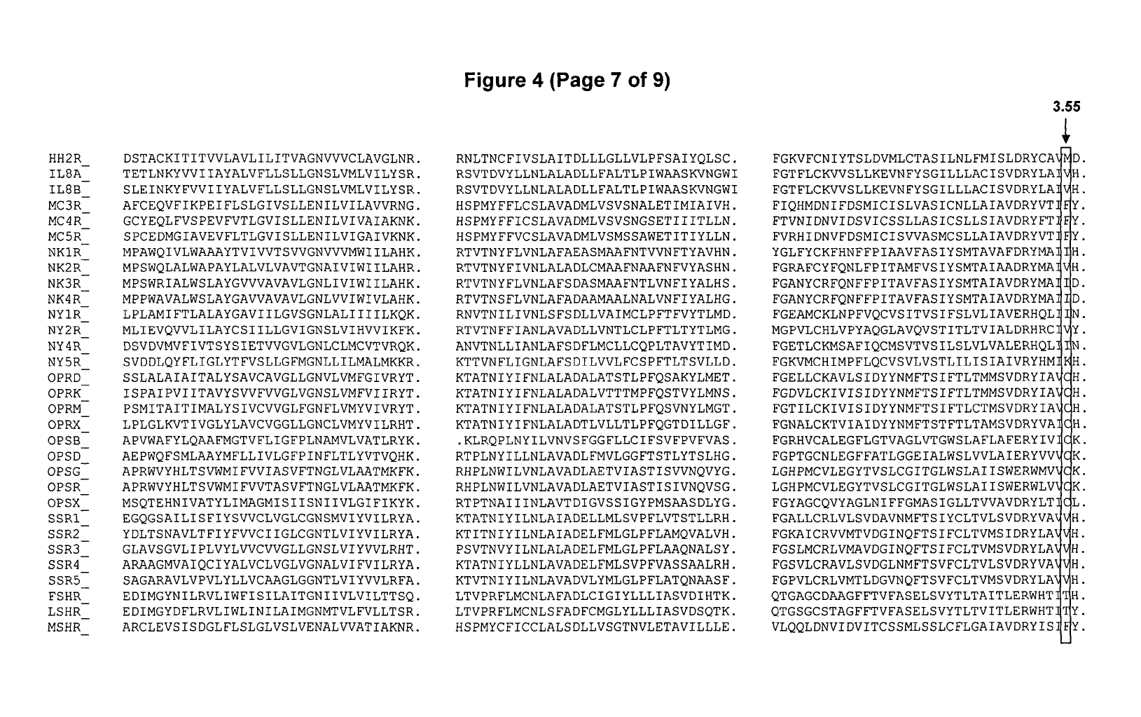

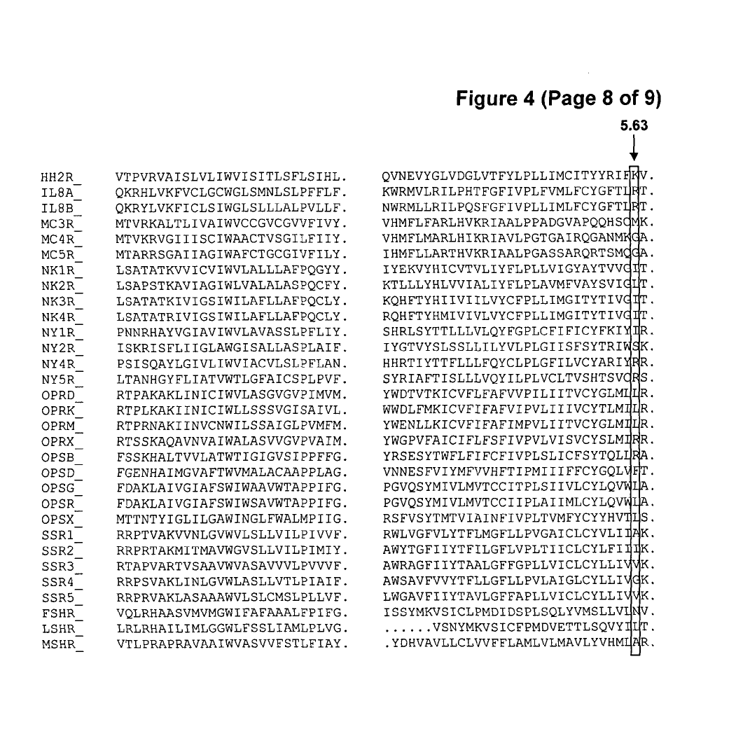

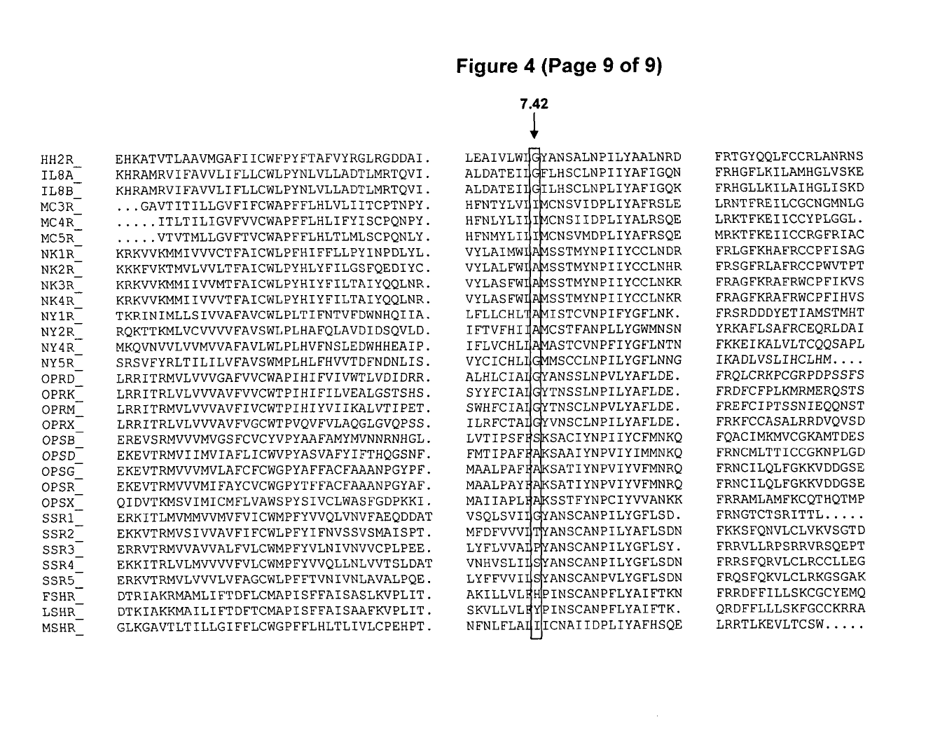

The amino acid sequences (and the nucleotide sequences of the cDNAs which encode them) of many GPCRs are readily available, for example by reference to GenBank. In particular, Foord et al supra gives the human gene symbols and human, mouse and rat gene IDs from Entrez Gene (http://www.ncbi.nlm.nih.gov/entrez). It should be noted, also, that because the sequence of the human genome is substantially complete, the amino acid sequences of human GPCRs can be deduced therefrom. FIG. 4 lists the amino acid sequences of various Class 1 GPCRs.

Although the parent GPCR may be any Class 1 GPCR, it is particularly preferred if it is a eukaryotic GPCR, that is the cDNA or gene encoding the GPCR is a eukaryotic cDNA or gene. For example, it is particularly preferred if the parent GPCR is a vertebrate GPCR such as a GPCR from a mammal. It is particularly preferred if the parent GPCR is from rat, mouse, rabbit or dog or non-human primate or human.

It is appreciated that the amino acid sequence defining the parent GPCR need not be an amino acid sequence defining the naturally occurring protein. Conveniently, it may define an engineered version which is capable of expression in a suitable host organism, such as in bacteria, yeast, insect cells or in mammalian cells. The amino acid sequence defining the parent GPCR may be an amino acid sequence defining a truncated form of the naturally occurring protein (truncated at either or both ends), or an amino acid sequence defining a fusion, either to the naturally occurring protein or to a fragment thereof, or an amino acid sequence that contains mutations compared to the naturally-occurring sequence. Alternatively or additionally, the amino acid sequence defining the parent GPCR, compared to a naturally-occurring GPCR, may be modified in order to improve, for example, solubility or proteolytic stability (eg by truncation, deletion of loops, mutation of glycosylation sites or mutation of reactive amino acid side chains such as cysteine). In any event, it will be appreciated that the amino acid sequence defining the parent GPCR is one that defines a GPCR that is able to bind to a ligand. The ligand may bind to the naturally occurring GPCR, or to a mutant thereof, or to a derivative of the naturally occurring GPCR or mutant thereof. By ‘derivative’ we include the meaning of a GPCR which compared to the naturally occurring GPCR has been chemically modified, for example by attachment of any chemical moiety to one or more amino acid side chains, or by the insertion of any chemical moiety within the amino acid sequence, but which derivative retains the ability to bind to a ligand.

Conveniently, the amino acid sequence defining a parent GPCR is one that defines a GPCR which, on addition of an appropriate ligand, can affect any one or more of the downstream activities which are commonly known to be affected by activation of G proteins, or other pathways independent of G proteins such as those which include arrestins. For example, where the parent GPCR is a 7-TMR that can signal independently of a G protein (e.g. smoothened or a mutant GPCR which has lost G protein signalling ability but retains signalling to other pathways), the amino acid sequence may be one that defines a parent GPCR which, on addition of an appropriate ligand, activates a G protein independent signalling pathway.

By the “3.55 amino acid residue”, we mean the amino acid residue at position 3.55 as defined by the Ballesteros numbering system (Ballesteros J A, Weinstein H. Integrated methods for the construction of three dimensional models and computational probing of structure-function relations in G-protein coupled receptors, Methods Neurosci 1995; 25:366-428). This is a general numbering scheme that applies to all rhodopsin-like GPCRs (Class 1 GPCRs). Each residue is identified by the transmembrane helix (TM) number (1 to 7), the most conserved residue within each TM is assigned the number ‘50’, and the positions of all other residues are numbered relative to the most conserved amino acid in each TM segment. Thus, amino acid residue 3.55 corresponds to the residue in TM3 that is 5 residues after the most conserved residue in TM2. The reference residue (i.e. number 50) in each TM has been assigned and they correspond to the following rhodopsin residues: TM1 N55, TM2 D83, TM3 R135, TM4 W161, TM5 P215, TM6 P267 and TM7 P303. The purpose of the Ballesteros numbering system is to facilitate identification of the homologous residues between different GPCRs using rhodopsin alignment as a guide.

Thus, the position of the 3.55 amino acid residue can be identified by locating the residue that is the most conserved residue in TM3 (i.e. number 50) and counting five residues after. In the human adenosine A_{2A} receptor, residue 3.55 corresponds to arginine 107. FIG. 4 provides an alignment of Class 1 GPCRs and shows the position of the 3.55 amino acid residue for each GPCR.

Generally, the 3.55 amino acid residue is any of threonine, valine, cysteine, phenylalanine or arginine. It is notable that although a basic amino acid residue is relatively uncommon at position 3.55 (˜8% Class 1 GPCRs), basic amino acids are more commonly found at position 3.56 (˜20% Class 1 GPCRs) and at position 3.57 (˜45% Class 1 GPCRs).

By the “5.63 amino acid residue”, we mean the amino acid residue at position 5.63 as defined by the Ballesteros numbering system. Accordingly, amino acid residue 5.63 corresponds to the residue in TM5 that is 13 residues after the most conserved residue in TM5.

Thus, the position of the 5.63 amino acid residue can be identified by locating the residue that is the most conserved residue in TM5 (i.e. number 50) and counting thirteen residues after. In the human adenosine Aₘ receptor, residue 5.63 corresponds to leucine 202. FIG. 4 provides an alignment of Class 1 GPCRs and shows the position of the 5.63 amino acid residue for each GPCR.

Generally, the 5.63 amino acid residue is any of arginine, lysine, isoleucine valine or leucine. It is notable that ˜14% Class 1 GPCRs have a leucine residue at position 5.63, ˜7% have an isoleucine residue, ˜7% have a valine residue, ˜28% have an arginine residue and ˜11% have a lysine residue.

By the “7.42 amino acid residue”, we mean the amino acid residue at position 7.42 as defined by the Ballesteros numbering system. Accordingly, amino acid residue 7.42 corresponds to the residue in TM7 that is eight residues before the most conserved residue in TM7.

Thus, the position of the 7.42 amino acid residue can be identified by locating the residue that is the most conserved residue in TM7 (i.e. number 50) and counting eight residues before. In the human adenosine A_{2A} receptor, residue 7.42 corresponds to serine 277. FIG. 4 provides an alignment of class 1 GPCRs and shows the position of the 7.42 amino acid residue for each GPCR.

Generally, the 7.42 amino acid residue is any of glycine, alanine, serine or threonine. It is notable that ˜44% Class 1 GPCRs have a glycine residue at position 7.42, ˜34% have an alanine residue and ˜8% have a serine residue. However, in the adenosine receptor family a serine residue at position 7.42 is highly conserved. For example the adenosine A_{2A}, A_{2B} and A₃ receptors each contain serine at position 7.42 and the adenosine A₁ receptor contains the closely related threonine.

Any suitable method may be used to align two polypeptide sequences, including but not limited to those described in Computational Molecular Biology (A. M. Lesk, ed., Oxford University Press 1988); Biocomputing: Informatics and Genome Projects (D. W. Smith, ed., Academic Press 1993); Computer Analysis of Sequence Data (Part 1, A. M. Griffin and H. G. Griffin, eds., Humana Press 1994); G. von Heinje, Sequence Analysis in Molecular Biology (Academic Press 1987); Sequence Analysis Primer (M. Gribskov and J. Devereux, eds., M. Stockton Press 1991); and Carillo et al., SIAMJ. Applied Math. 48: 1073 (1988). Preferred methods to align polypeptides are designed to give the largest match between the sequences tested. Methods to determine identity and similarity are codified in publicly available computer programs.

Preferred computer program methods to align polypeptide sequences and to determine identity and similarity between two sequences include, but are not limited to, the GCG program package, including GAP (Devereux et al., Nuc. Acids Res. 12: 387 (1984); Genetics Computer Group, University of Wisconsin, Madison, Wis.), BLASTP, BLASTN, and FASTA (Atschul et al., J. Mol. Biol. 215: 403-10 (1990)). The BLAST X program is publicly available from the National Center for Biotechnology Information (NCBI) and other sources (Altschul et al., BLAST Manual (NCB NLM NIH, Bethesda, Md.); Altschul et al., 1990, supra). The well-known Smith Waterman algorithm may also be used to determine identity.

By way of example, using the computer algorithm GAP (Genetics Computer Group), two polypeptides for which the percent sequence identity is to be determined are aligned for optimal matching of their respective amino acids (the “matched span,” as determined by the algorithm). A gap opening penalty (which is calculated as 3× the average diagonal; the “average diagonal” is the average of the diagonal of the comparison matrix being used; the “diagonal” is the score or number assigned to each perfect amino acid match by the particular comparison matrix) and a gap extension penalty (which is usually 0.1× the gap opening penalty), as well as a comparison matrix such as PAM 250 or BLOSUM 62 are used in conjunction with the algorithm. A standard comparison matrix (see Dayhoff et al., 5 Atlas of Protein Sequence and Structure (Supp. 3 1978) for the PAM250 comparison matrix; see Henikoff et al., Proc. Natl. Acad. Sci. USA 89: 10915-19 (1992) for the BLOSUM 62 comparison matrix) is also used by the algorithm. Preferred parameters for polypeptide sequence comparison include the following: Algorithm: Needleman and Wunsch, J. Mol. Biol. 48: 443-53 (1970). Comparison matrix: BLOSUM from Henikoff et al., Proc. Natl. Acad. Sci. U.S.A. 89: 10915-19 (1992) Gap Penalty: 12 Gap Length Penalty: 4 Threshold of Similarity: 0 The GAP program is useful with the above parameters. The aforementioned parameters are the default parameters for polypeptide comparisons (along with no penalty for end gaps) using the GAP algorithm.

By a “window of i plus or minus 5 residues” we include the 3.55 amino acid residue and the five amino acid residues before and the five amino acid residues after, the 3.55 amino acid residue. Thus, within a window of i plus or minus 5 residues, one or more of the i−5, i−4, i−3, i−2, i−1, i, i+1, i+2, i+3, i+4 and i+5 amino acid residues may be mutated, for example any 1, 2, 3, 4, 5, 6, 7, 8, 9, 10 or all 11 of the i−5, i−4, i−3, i−2, i−1, i, i+1, i+2, i+3, i+4 and i+5 amino acid residues may be mutated.

In an embodiment, one or more mutations are made in an amino acid sequence that defines a parent GPCR within a window of i plus or minus 4 residues, where i is the position of amino acid residue 3.55, in the parent GPCR, to provide one or more mutants of the parent GPCR with increased stability. By a “window of i plus or minus 4 residues” we include that 3.55 amino acid residue and the four amino acid residues before and the four amino acid residues after, the 3.55 amino acid residue. Thus, within a window of i plus or minus 4 residues, one or more of the i−4, i−3, i−2, i+1, i+2, i+3 and i+4 amino acid residues may be mutated, for example any 1, 2, 3, 4, 5, 6, 7, 8 or all 9 of the i−4, i−3, i−2, i−1, i, i+1, i+2, i+3 and i+4 amino acid residues may be mutated.

In an embodiment, one or more mutations are made in an amino acid sequence that defines a parent GPCR within a window of i plus or minus 3 residues, where i is the position of amino acid residue 3.55 in the parent GPCR, to provide one or more mutants of the parent GPCR with increased stability. By a “window of i plus or minus 3 residues” we include the 3.55 amino acid residue and the three amino acid residues before and the three amino acid residues after, the 3.55 amino acid residue. Thus, within a window of i plus or minus 3 residues, one or more of the i−3, i−2, i−1, i, i+1, i+2 and i+3 amino acid residues may be mutated, for example any 1, 2, 3, 4, 5, 6 or all 7 of the i−3, i−2, i−1, i, i+1, i+2 and i+3 amino acid residues may be mutated.

In an embodiment, one or more mutations are made in an amino acid sequence that defines a parent GPCR within a window of i plus or minus 2 residues, where i is the position of amino acid residue 3.55 in the parent GPCR to provide one or more mutants of the parent GPCR with increased stability. By a “window of i plus or minus 2 residues” we include the 3.55 amino acid residue and the two amino acid residues before and the two amino acid residues after, the 3.55 amino acid residue. Thus, within a window of i plus or minus 2 residues, one or more of the i−2, i−1, i, i+1 and i+2 amino acid residues may be mutated, for example any 1, 2, 3, 4 or all 5 of the i−2, i−1, i, i+1 and i+2 amino acid residues may be mutated.

In another embodiment, one or more mutations are made in an amino acid sequence that defines a parent GPCR within a window of i plus or minus 1 residue, where i is the position of amino acid residue 3.55 in the parent GPCR to provide one or more mutants of the parent GPCR with increased stability. By a “window of i plus or minus 1 residue” we include the 3.55 amino acid residue and the amino acid residue before and the amino acid residue after, the 3.55 amino acid residue. Thus, within a window of i plus or minus 1 residue, one or more of the i−1, i and i+1 amino acid residues may be mutated, for example any 1, 2 or all 3 of the i−1, i and i+1 amino acid residues may be mutated.

By a “window of i minus 2 to i residues” we include the 5.63 amino acid residue and up to two amino acid residues before the 5.63 amino acid residue. Thus, within a window of i minus 2 to i residues, one or more of the i−2, i−1 and i amino acid residues may be mutated, for example any 1 or 2 or all 3 of the i−2, i−1 and i amino acid residues may be mutated.

In an embodiment, one or more mutations are made in an amino acid sequence that defines a parent GPCR within a window of i minus 1 to i residues, where i is the position of amino acid residue 5.63 in the parent GPCR, to provide one or more mutants of the parent GPCR with increased stability. By a “window of i minus 1 to i residues” we include that 5.63 amino acid residue and the amino acid residue before the 5.63 amino acid residue. Thus, one or both of the i−1 and i amino acid residues may be mutated.

By a “window of i minus 4 to i plus 1 residues” we include the 7.42 amino acid residue and the four amino acid residues before and the one amino acid residue after the 7.42 amino acid residue. Thus, within a window of i minus 4 to i plus 1 residues, one of more of the i−4, i−3, i−2, i−1, i and i+1 amino acid residues may be mutated, for example any 1, 2, 3, 4, 5 or all 6 of the i−4, i−3, i−2, i−1, i and i+1 amino acid residues may be mutated.

In an embodiment, one or more mutations are made in an amino acid sequence that defines a parent GPCR within a window of i minus 3 to i plus 1 residues, where i is the position of amino acid residue 7.42, in the parent GPCR, to provide one or more mutants of the parent GPCR with increased stability. By a “window of i minus 3 to i plus 1 residues” we include the 7.42 amino acid residue and the three amino acid residues before and the one amino acid residue after, the 7.42 amino acid residue. Thus, within a window of i minus 3 to i plus 1 residue, one or more of the i−3, i−2, i−1, i and i+1 amino acid residues may be mutated, for example any 1, 2, 3, 4 or all 5 of the i−3, i−2, i−1, i and i+1 amino acid residues may be mutated.

In an embodiment, one or more mutations are made in an amino acid sequence that defines a parent GPCR within a window of i minus 2 to i plus 1 residues, where i is the position of amino acid residue 7.42 in the parent GPCR, to provide one or more mutants of the parent GPCR with increased stability. By a “window of i minus 2 to i plus 1 residues” we include the 7.42 amino acid residue and the two amino acid residues before and the one amino acid residue after, the 7.42 amino acid residue. Thus, within a window of i minus 2 to i plus 1 residue, one or more of the i−2, i−1, i and i+1 amino acid residues may be mutated, for example any 1, 2, 3 or all 4 of the i−2, i−1, i and i+1 amino acid residues may be mutated.

In an embodiment, one or more mutations are made in an amino acid sequence that defines a parent GPCR within a window of i plus or minus 1 residue, where i is the position of amino acid residue 7.42 in the parent GPCR to provide one or more mutants of the parent GPCR with increased stability. By a “window of i plus or minus 1 residue” we include the 7.42 amino acid residue and the amino acid residue before and the amino acid residue after, the 7.42 amino acid residue. Thus, within a window of i plus or minus 1 residue, one or more of the i−1, i and i+1 amino acid residues may be mutated, for example any 1, 2 or all 3 of the i−1, i and i+1 amino acid residues may be mutated.

Changes to a single amino acid within the GPCR may increase the stability of the protein compared to the parent protein. Thus, in one embodiment of the method of producing a mutant GPCR with increased stability, a single amino acid residue of the parent protein is changed in the mutant protein. However, it is appreciated that a further increase in stability may be obtained by changing more than one of the amino acids of the parent protein. For example, more than one amino acid within any of the windows mentioned above (i.e. windows where i is 3.55, 5.63 or 7.42) may be changed in the parent GPCR, and/or more than one amino acid within two or more of the windows mentioned above (e.g. two or more of a window where i is 3.55, a window where i is 5.63 or a window where i is 7.42) may be changed in the parent GPCR.

Typically when producing a mutant GPCR with increased stability, the mutant GPCR contains, compared to the parent protein, from 1 to 11 changed amino acids, preferably from 1 to 8 or from 1 to 6, such as 2 to 6, for example 2, 3, 4, 5 or 6 changed amino acids. However, it is appreciated that the total number of mutations required to confer increased stability may be more than this, and will ultimately vary from receptor to receptor, depending on various factors such as the intrinsic stability of the parent receptor.

Mutations can be made in an amino acid sequence defining a parent GPCR using any suitable technique known in the art. For example, conventional site-directed mutagenesis may be employed, or polymerase chain reaction-based procedures may be used, such that particular amino acid residues are independently replaced with other amino acid residues. Molecular biological methods for cloning and engineering genes and cDNAs, for mutating DNA, and for expressing polypeptides from polynucleotides in host cells are well known in the art, as exemplified in “Molecular cloning, a laboratory manual”, third edition, Sambrook, J. & Russell, D. W. (eds), Cold Spring Harbor Laboratory Press, Cold Spring Harbor, N.Y.

Typically, making one or more mutations in the amino acid sequence that defines a parent GPCR comprises replacing one or more amino acids by Ala, although it may be replaced by any other amino acid. For example, if a particular amino acid within any of the windows described above is Ala, it may conveniently be replaced by Leu. Alternatively, the amino acid may be replaced by Gly for example, which may allow a closer packing of neighbouring helices that may lock the protein in a particular conformation. If the amino acid is Gly, it may conveniently be replaced by Ala for example.

Although the amino acid used to replace a given amino acid at a particular position is typically a naturally occurring amino acid, typically an “encodeable” amino acid, it may be a non-natural amino acid (in which case the protein is typically made by chemical synthesis or by use of non-natural amino-acyl tRNAs). An “encodeable” amino acid is one which is incorporated into a polypeptide by translation of mRNA. It is also possible to create non-natural amino acids or introduce non-peptide linkages at a given position by covalent chemical modification, for example by post-translational treatment of the protein or semisynthesis. These post-translational modifications may be natural, such as phosphorylation, glycosylation or palmitoylation, or synthetic or biosynthetic.

The inventors have shown that mutating any of residues 3.55, 5.63 and 7.42 increases the stability of a particular conformation (eg antagonist). In other words the stability of that conformation is increased relative to the stability of that same conformation in the parent GPCR. Thus, the method of the invention may be considered to be a method for producing mutants of a GPCR which have increased stability of a particular conformation, for example they may have increased conformational thermostability. The method of the invention may therefore be used to create stable, conformationally locked GPCRs by mutagenesis. The mutant GPCRs are effectively purer forms of the parent molecules in that a much higher proportion of them occupies a particular conformational state. In an embodiment, the one or more mutants of the parent GPCR have increased stability in an agonist or antagonist conformation. It is appreciated that the method of the invention may also be considered to be a method for producing mutant GPCRs which are more tractable to crystallisation.

Conveniently, the method of the first aspect of the invention is performed and the stability of the resulting one or more mutants assessed. Methods for assessing the stability of GPCRs are known in the art and are described, for example, in WO 2008/114020 and in WO 2009/071914. Preferably, it is determined whether the resulting one or more mutants when residing in a particular conformation have increased stability with respect to binding a ligand (the ligand being one which binds to the parent GPCR when the parent GPCR is residing in a particular conformation), compared to the stability of the parent GPCR when residing in the same particular conformation with respect to binding that ligand. It is appreciated that the comparison of stability of the one or more mutants is made by reference to the parent molecule under the same conditions.

Since there are potentially thousands of mutations that can be screened in a GPCR for increased stability, it is advantageous to target particular mutations which are known to be important in conferring stability. Therefore, it will be appreciated that the method of the first aspect of the invention may also be used as a method of selecting mutant GPCRs with increased stability. In particular, carrying out the method of the first aspect of the invention can be used to target mutations to particular amino acid residues (e.g. within a window of i plus or minus 5 residues where i is the position of amino acid residue 3.55, or its equivalent as the case may be in the parent GPCR; or within a window of i minus 2 to i residues where i is the position of amino acid residue 5.63, or its equivalent as the case may be in the parent GPCR; or within a window of i minus 4 to i plus 1 residues where i is the position of amino acid residue 7.42, or its equivalent as the case may be in the parent GPCR; although it is appreciated that stabilising mutations may be located outside these windows). The resulting one or more mutants can then be tested for increased stability, and those that have increased stability selected.

It is appreciated that the method of the first aspect of the invention may be repeated, for example once the stability of the resulting one or mutants are assessed, with the resulting one or more mutants generated in the first round becoming the parent GPCR in a subsequent round. Thus, the method can be used in an iterative way by, for example, carrying out a method to identify single mutations with increased stability, combining those mutations in a single mutant GPCR, which then becomes the parent GPCR that is mutated in a subsequent round.

For example, in an embodiment of the first aspect of the invention, the one or more mutations in the amino acid sequence that defines a parent GPCR may be made within progressively increasing window sizes. Typically, a small window size is used first, and the stabilising mutations from that sequence subset identified, followed by scanning further residues at increasing window sizes until the desired number of mutations have been found. Thus in a first round, one or more mutations may be made in the amino acid sequence that defines a parent GPCR within a window of i plus or minus 1 residue, wherein i is the position of amino acid residue 3.55, or its equivalent as the case may be, in the parent GPCR. The stability of the resulting mutants may be assessed and those mutants that have an increased stability compared to the parent GPCR selected. The method may then be repeated by making one or more mutations in the amino acid sequence that defines a parent GPCR (corresponding to a mutant selected in the first round) within a window or windows of i plus or minus 2 residues, and so on.

The mutant GPCR may be one which has increased stability to any denaturant or denaturing condition such as to any one or more of heat, a detergent, a chaotropic agent or an extreme of pH.

In relation to an increased stability to heat (ie thermostability), this can readily be determined by measuring ligand binding or by using spectroscopic methods such as fluorescence, CD or light scattering at a particular temperature. Typically, when the GPCR binds to a ligand, the ability of the GPCR to bind that ligand at a particular temperature may be used to determine thermostability of the mutant. It may be convenient to determine a “quasi Tₘ” ie the temperature at which 50% of the receptor is inactivated under stated conditions after incubation for a given period of time (eg 30 minutes). Mutant GPCRs of higher thermostability have an increased quasi Tm compared to their parents. Alternatively, thermostability can be assessed by measuring stability at a given temperature as a function of time. For example, the length of time at a given temperature by which the level of ligand binding falls to 50% of the level of ligand binding at time zero may be determined (Shibata et al., 2009 J Mol Biol). In either case however, it is appreciated that temperature is the denaturant.

In relation to an increased stability to a detergent or to a chaotrope, typically the GPCR is incubated for a defined time in the presence of a test detergent or a test chaotropic agent and the stability is determined using, for example, ligand binding or a spectroscopic method as discussed above.

In relation to an extreme of pH, a typical test pH would be chosen (eg in the range 4.5 to 5.5 (low pH) or in the range 8.5 to 9.5 (high pH).

Because relatively harsh detergents are used during crystallisation procedures, it is preferred that the mutant GPCR is stable in the presence of such detergents. The order of “harshness” of certain detergents is DDM, C₁₁→C₁₀→C₉→C₈ maltoside or glucoside, lauryldimethylamine oxide (LDAO) and SDS. It is particularly preferred if the mutant GPCR is more stable to any of C₉ maltoside or glucoside, C₈ maltoside or glucoside, LDAO and SDS, and so it is preferred that these detergents are used for stability testing.

Because of its ease of determination, it is preferred that the mutant GPCR has increased thermostability compared to its parent protein. It will be appreciated that heat is acting as the denaturant, and this can readily be removed by cooling the sample, for example by placing on ice. It is believed that thermostability may also be a guide to the stability to other denaturants or denaturing conditions. Thus, increased thermostability is likely to translate into stability in denaturing detergents, especially those that are more denaturing than DDM, eg those detergents with a smaller head group and a shorter alkyl chain and/or with a charged head group. We have found that a thermostable GPCR is also more stable towards harsh detergents.

When an extreme of pH is used as the denaturing condition, it will be appreciated that this can be removed quickly by adding a neutralising agent. Similarly, when a chaotrope is used as a denaturant, the denaturing effect can be removed by diluting the sample below the concentration in which the chaotrope exerts its chaotropic effect.

In a further embodiment of the method of the first aspect of the invention, it is determined whether the mutant GPCR is able to couple to a G protein or another protein known to interact with a GPCR, for example a signalling protein such as arrestin or a GPCR kinase. Preferably, it is also determined whether the mutant GPCR is able to bind a plurality of ligands of the same class (eg agonist or antagonist), with a comparable spread and/or rank order of affinity as the parent GPCR.

Preferably, the parent GPCR is any of an adenosine receptor, a serotonin receptor, a β-adrenergic receptor, a neurotensin receptor or a muscarinic receptor. More preferably, the parent GPCR is an adenosine receptor such as any of an A_{2A} receptor, an A_{2B} receptor, an A₁ receptor or an A₃ receptor.

### Mutant Adenosine Receptor

Adenosine receptors are well known in the art. They share sequence homology to each other and bind to adenosine. The invention provides particular mutant adenosine receptors and methods for producing them.

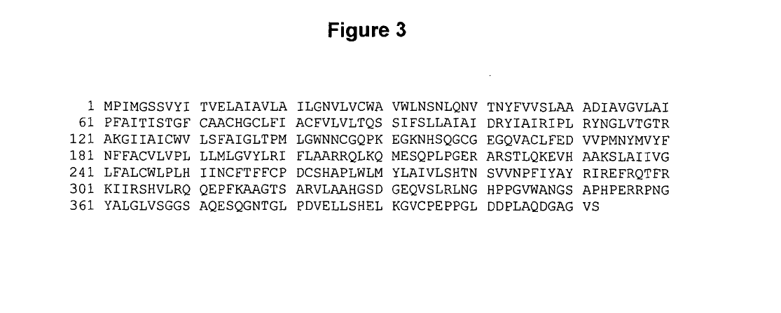

In a particularly preferred embodiment, the mutant GPCR with increased stability relative to its parent GPCR is a mutant adenosine receptor which, when compared to the corresponding parent receptor, has a different amino acid at a position which corresponds to one or more of Arg 107, Leu 202 and Ser 277 according to the numbering of the human adenosine A_{2A} receptor as set out in FIG. 3.

The mutant adenosine receptor may be a mutant of any adenosine receptor provided that it is mutated at a position which corresponds to any one or more of Arg 107, Leu 202 and Ser 277 according to the numbering of the human adenosine A_{2A} receptor as set out in FIG. 3.

It is particularly preferred if the mutant GPCR is one that has at least 20% amino acid sequence identity when compared to the given human adenosine A_{2A} receptor whose sequence is set out in FIG. 3, as determined using MacVector and CLUSTALW (Thompson et al (1994) Nucl. Acids Res. 22, 4673-4680). More preferably, the mutant GPCR has at least 30% or at least 40% or at least 50%, or at least 60% amino acid sequence identity. There is generally a higher degree of sequence conservation at the adenosine binding site.

As is described in Example 1 below, replacement of Arg 107, Leu 202 and Ser 277 leads to an increase in thermostability when measured with the inverse agonist ZM-241,385. For example, individual replacement of Arg 107, Leu 202 and Ser 277 increases the Tₘ of the A_{2A}-StaR1 by 2.1° C., 7.1° C. and 3.8° C., respectively (see Table 2).

Thus, the mutant GPCR may be a mutant human adenosine A_{2A} receptor in which, compared to its parent, one or more of Arg 107, Leu 202 and Ser 277 have been replaced by another amino acid residue. The mutant GPCR may also be a mutant adenosine receptor from another source in which one or more corresponding amino acids in the parent receptor are replaced by another amino acid residue. For the avoidance of doubt, the parent may be an adenosine receptor which has a naturally-occurring sequence, or it may be a truncated form or it may be a fusion, either to the naturally-occurring protein or to a fragment thereof, or it may contain mutations compared to the naturally-occurring sequence, provided that it retains ligand-binding ability.

By “corresponding amino acid residue” we include the meaning of the amino acid residue in another adenosine receptor which aligns to the given amino acid residue in human adenosine A_{2A} receptor when the human adenosine A_{2A} receptor and the other adenosine receptor are compared using MacVector and CLUSTALW.

Other human adenosine receptors include adenosine A_{2B}, A₃ and A₁ receptors. Thus the mutant GPCR may be a mutant adenosine A_{2B} receptor or a mutant adenosine A₃ receptor or a mutant adenosine A₁ receptor in which one or more amino acids corresponding to any of Arg 107, Leu 202 and Ser 277 has been replaced by another amino acid. For example in the adenosine A₁ receptor, the amino acid residues corresponding to Arg 107, Leu 202 and Ser 277 in the A_{2A} receptor, are Lys 110, Tyr 205 and Thr 277 respectively and so the mutant GPCR may be a mutant adenosine A₁ receptor in which one or more of Lys 110, Tyr 205 and Thr 277 have been replaced by another amino acid.

Although in the preferred embodiment of Example 1, the mutant GPCR is a human adenosine A_{2A} receptor in which, compared to its parent, Arg 107, Leu 202 and Ser 277 have been replaced by an alanine amino acid residue, it is of course appreciated that the mutant GPCR may be a human adenosine A_{2A} receptor which, when compared to the corresponding parent receptor has an amino acid residue other than alanine at each of positions Arg 107, Leu 202 and Ser 277 according to the numbering of the human adenosine A_{2A} receptor as set out in FIG. 3. Similarly, the mutant GPCR may be any other GPCR which, when compared to the corresponding parent receptor has a different amino acid at a position which corresponds to one or more of Arg 107, Leu 202 and Ser 277 according to the numbering of the human adenosine A_{2A} receptor as set out in FIG. 3.

It is likewise appreciated that the mutant GPCR may be one in which, compared to its parent, has a different amino acid at one or more positions in the windows defined above other than at positions which correspond to one or more of Arg 107, Leu 202 and Ser 277 according to the numbering of the human adenosine A₂, receptor as set out in FIG. 3.

Although in the preferred embodiment of Example 1, the mutant GPCR is a human adenosine A2a receptor with eight stabilising mutations, namely A54L^{2.52}, T88A^{3.36}, K122A^{4.43}, V239A^{6.41}, R107A^{3.55}, L202A^{5.63}, L235A^{6.37}, and S277A^{7.42}, which include stabilising mutations at positions Arg 107, Leu 202 and Ser 277, it will of course be appreciated that the mutant GPCR may be any one as defined according to the first aspect of the invention, including a human adenosine A2a receptor, but which when compared to the corresponding parent receptor does not have a leucine residue at a position which corresponds to Ala 54, an alanine at a position which corresponds to Thr 88, an alanine at a position which corresponds to Lys 122, an alanine at a position which corresponds to Val 239 and an alanine at a position which corresponds to Leu 235, an alanine at a position which corresponds to Arg 107, an alanine at a position which corresponds to Leu 202, and an alanine at a position which corresponds to Ser 277, according to the to the numbering of the human adenosine A_{2A} receptor as set out in FIG. 3.

A second aspect of the invention provides a mutant GPCR with increased stability relative to its parent GPCR obtainable by the method of the first aspect of the invention.

The inventors have demonstrated that making mutations in the amino acid sequence that defines a parent GPCR within one of the windows mentioned above can provide mutants of the parent GPCR that have increased stability. Thus, it is appreciated that the mutant GPCRs of the second aspect of the invention will have an extended lifetime, relative to its parent, under destabilising conditions.

Accordingly, a third aspect of the invention provides a mutant GPCR which, when compared to a Class 1 parent GPCR, has one or more mutations in the amino acid sequence defining the parent GPCR, wherein (i) the one or more mutations are located within a window of i plus or minus 5 residues, where i is the position of amino acid residue 3.55 in the parent GPCR and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, to provide one or more mutants of the parent GPCR with increased stability.

It also appreciated that the invention allows for the production of compositions comprising mutant GPCRs, characterised in that the mutant GPCR is exposed to a destabilising condition. Such compositions have various applications, for example in crystallisation, drug screening, bioassay and biosensor applications.

Thus, a fourth aspect of the invention provides a composition comprising a mutant GPCR which, when compared to a Class 1 parent GPCR, has one or more mutations in the amino acid sequence defining the parent GPCR, wherein (i) the one or more mutations are located within a window of i plus or minus 5 residues, where i is the position of amino acid residue 3.55 in the parent GPCR, and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, to provide one or more mutants of the parent GPCR with increased stability, characterised in that the mutant GPCR is exposed to a destabilising condition effective to destabilise a parent GPCR to a greater extent than the mutant GPCR.

By “destabilising condition” we include any condition which is capable of shifting the equilibrium of a population of GPCR proteins from the folded native state in a membrane to the unfolded state. In this way, the proportion of GPCR proteins existing in the unfolded state is increased and the proportion existing in the folded native state in a membrane is decreased.

By “population” we include a plurality of the same specific type of GPCR, as opposed to a mixture of different GPCRs. For example, the population may comprise at least 2, 5, 10, 50, 100, 200, 500, 1000, 5000, 10000, 100000, 10⁶, 10⁷, 10⁸, 10⁹, 10¹⁰, 10¹¹, 10¹², 10¹³ or 10¹⁴ GPCR molecules. Preferably, the population may comprise GPCR 10⁹ and 10¹² GPCR molecules.

In the folded native state in a membrane, GPCRs exhibit a biological activity, for example a binding activity or a signalling pathway modulation activity. Upon increasing exposure to a destabilising condition as described above, the equilibrium shifts further towards the unfolded state and an increasingly higher proportion of the GPCRs exist in the unfolded state. This change in structure from a folded to an unfolded state leads to a detectable change in the structure of the GPCR population. Moreover, this change in structure may lead to a detectable decrease in a biological activity of the GPCR population.

Accordingly in one embodiment, the destabilising condition is one that is effective to bring about a significant perturbation in the structure of a GPCR population compared to the structure of that population in the absence of the destabilising condition.

By a “significant perturbation in the structure of a GPCR population”, we mean a perturbation which, when assessed relative to the statistical variation of the measurements used to detect the perturbation, would arise by chance in less than 1 in 10 measurements, more preferably 1 in 20 measurements and even more preferably 1 in 50 or 1 in 100 measurements.

Various methods to probe protein structure are known in the art and any suitable method may be used. For example, structural perturbations may be assayed by probing conformation directly e.g. with covalently attached fluorescent labels or esr spin labels, or by measuring the accessibility of native or deliberately introduced amino acid side chains within the protein population (Hubbell, W. L. et al., Adv. Protein. Chem. 63, 243-290 (2003); Baneres, J. L. et. al., J. Biol. Chem. 280, 20253-20260 (2005); Kobilka, B. K. and Deupi, X. Trends. Pharmacol. Sci. 28, 397-406 (2007)). For example, changes in fluorescence spectra, can be a sensitive indicator of protein unfolding, either by use of intrinsic tryptophan fluorescence or the use of the thiol specific fluorochrome N-[4-(7-diethylamino-4-methyl-3-coumarinyl)phenyl]maleimide (cpm) that becomes fluorescent upon reacting with cysteine residues exposed upon protein unfolding (Alexandrov et al (2008) Structure 16: 351-359). Proteolytic stability, deuterium/hydrogen exchange measured by mass spectrometry or nuclear magnetic resonance spectroscopy, blue native gels, capillary zone electrophoresis, circular dichroism (CD) or linear dichroism (LD) spectra and light scattering may also be used to measure structural perturbation by loss of signals associated with secondary or tertiary structure.

In another embodiment, the destabilising condition is one that is effective to bring about a significant reduction in a biological activity of a GPCR population (e.g. binding activity or signalling pathway modulation activity), compared to the level of the same activity in the absence of the destabilising condition. For example, the agent may be one that reduces the biological activity of a GPCR population to 90-10%, such as 70-30% or 60-40% of the level of the same activity when measured in the absence of the destabilising condition.

Depending upon the biological activity, it will be appreciated that the activity of the GPCR population may be measured using any suitable method known in the art.

By ‘binding activity’, we include binding to any binding partner that is known to bind to the GPCR. For example, the binding partner may be a ligand, for example one which causes the GPCR to reside in a particular conformation, or it may be an antibody, for example a conformational-specific antibody. Binding activity can be assessed using routine binding assays known in the art. Conveniently, the binding partner is detectably labelled, eg radiolabelled or fluorescently labelled. Alternatively, binding can be assessed by measuring the amount of unbound binding partner using a secondary detection system, for example an antibody or other high affinity binding partner covalently linked to a detectable moiety, for example an enzyme which may be used in a colorimetric assay (such as alkaline phosphatase or horseradish peroxidase). Biophysical techniques such as patch clamping, fluorescence correlation spectroscopy, fluorescence resonance energy transfer and analytical ultracentrifugation may also be used (as described in New, R. C., Liposomes: a practical approach. 1st ed.; Oxford University Press: Oxford, 1990, and Graham, J. M.; Higgins, J. A., Membrane Analysis. Springer-Verlag: New York, 1997.)

Where the biological activity is a signalling pathway modulating activity, the activity can be assessed by any suitable assay for the particular signalling pathway. The pathway may be downstream of G protein activation or may be independent of G protein activation. Activation of the G protein can be measured directly by binding of a guanine nucleotide such as radiolabelled GTP to the G protein (Johnson et al, Assay Drug Dev Technol. 2008 June; 6(3):327-37). Alternatively, binding of a signalling protein such as a G protein or an arrestin to the receptor may be measured by fluorescence resonance energy transfer (FRET) (Lohse et al, Adv Protein Chem. 2007; 74:167-88) or related assays such as bioluminescence resonance energy transfer (BRET) (Gales et al, Nat. Methods. 2005 March; 2(3):177-84) or an enzyme complementation assay (Zhao et al, J Biomol Screen. 2008 September; 13(8):737-47. Epub 2008). These assays are commonly available in kits for example from Perkin Elmer or CisBio or DiscoverX. The activity may be measured by using a reporter gene to measure the activity of the particular signalling pathway. By a reporter gene we include genes which encode a reporter protein whose activity may easily be assayed, for example β-galactosidase, chloramphenicol acetyl transferase (CAT) gene, luciferase or Green Fluorescent Protein (see, for example, Tan et al, 1996 EMBO J. 15(17): 4629-42). Several techniques are available in the art to detect and measure expression of a reporter gene which would be suitable for use in the present invention. Many of these are available in kits both for determining expression in vitro and in vivo. Alternatively, signalling may be assayed by the analysis of downstream targets. For example, a particular protein whose expression is known to be under the control of a specific signalling pathway may be quantified, or a secondary metabolite may be quantified. Protein levels in biological samples can be determined using any suitable method known in the art. For example, protein concentration can be studied by a range of antibody based methods including immunoassays, such as ELISAs, western blotting and radioimmunoassay or by the use of biosensors (Ponsioen et al EMBO Rep. 2004 December; 5(12):1176-80).

In an embodiment, the destabilising condition is any of heat, a detergent, a chaotropic agent such as guanidinium thiocyanate, an extreme of pH, an organic solvent, an aqueous solution or a membrane free environment.

For example, the destabilising condition may be a detergent, including for example, detergents that are of interest for subsequent crystallisation studies, for instance short chain-length detergents with a high CMC, such as C8-glucoside, C8-thioglucoside, C9-glucoside, C8-maltoside, C8-thiomaltoside, C9-maltoside, C9-thiomaltoside, Cymal 5, C8E5, or lauryl dimethylamine oxide. Short chain-length detergents are more likely to allow the formation of a 3-dimensional crystal lattice, and are easier to remove from receptor preparations by dialysis or other means than are long chain-length detergents with low CMCs.

It will also be appreciated that the destabilising condition may be any other amphiphilic molecule. For example, the destabilising condition may be any of amphipols, amphiphilic peptides such as mellitin, proteins such as apolipoproteins and their derivatives, organic solvents such as trifluoroethanol, dimethylformide, dimethylsulphoxide and chloroform/methanol mixtures, urea, a cylcodextrin, poly-ene antibiotics, guanidine hydrogenchloride, local anaesthetics and drugs such as procaine and chlorpromazine, polyols such as butane diol and heptane triol, short chain alcohols such as ethanol, propanol, isopropanol, butane diol and benzyl alcohol.

It will also be appreciated that the destabilising condition may be any aqueous solution.

It will further be appreciated that the destabilising condition may be a membrane free environment, such that the mutant GPCR exists in a form that is membrane free as discussed below.

In any event, the destabilising condition is one which is capable of shifting the equilibrium of a population of GPCR proteins from the folded native state in the membrane, to the unfolded state.

In one embodiment of the third and fourth aspects of the invention, the mutant GPCR is membrane free. By ‘membrane free’ we include the meaning of the mutant GPCR being substantially free of a membrane such as a lipid bilayer or a lipid monlayer. For example, the mutant GPCR may be in a form where it does not reside within a membrane, unlike when it does reside in a membrane when in the native folded state.

Given the increased stability of a mutant GPCR according to the invention, it is appreciated that the destabilising condition will destabilise the parent GPCR to a greater extent than the mutant GPCR, i.e. shift the equilibrium of a population of the parent GPCR from the folded native state to the unfolded state, further than it shifts the equilibrium of a population of the mutant GPCR from the folded native state in a membrane to the unfolded state.

Thus, when the parent GPCR manifests, for example, 50% of a biological activity when exposed to a destabilising condition, typically, the mutant GPCR with increased stability relative to the parent protein, will have at least 5%, 10%, 15%, 20%, 25%, 30%, 35%, 40%, 45% or 50% more biological activity than the parent protein when exposed to the destabilising condition, and more preferably at least 60%, 70%, 80%, 90% or 100% more activity, and yet more preferably at least 150% or 200% more activity.

Similarly, a mutant GPCR with increased stability relative to the parent GPCR will have a structure that is more similar to the folded-native state than the structure of the parent protein is to the folded-native state, when exposed to a destabilising condition.

In this way, the invention allows for mutant GPCRs that have increased stability compared to a parent GPCR when exposed to a destabilising condition, and for compositions comprising a mutant GPCR of the invention, characterised in that the mutant GPCR is exposed to a destabilising agent effective to destabilise a parent GPCR to a greater extent than the mutant GPCR. For example, the invention allows for a solubilised form of a mutant GPCR of the invention.

Preferences for the mutant GPCRs are as defined above with respect to the first aspect of the invention. For example, the mutant GPCR of the third aspect of the invention may be, or the composition of the fourth aspect of the invention may comprise, a mutant adenosine receptor which, when compared to the corresponding parent receptor, has a different amino acid at a position which corresponds to one or more of Arg 107, Leu 202 and Ser 277 according to the numbering of the human adenosine A_{2A} receptor as set out in FIG. 3. Preferably, the adenosine receptor has an amino acid sequence which is at least 20% identical to that of the human adenosine receptor whose sequence is set out in FIG. 3.

It is preferred that the mutant GPCR of the second or third aspect of the invention, or the mutant GPCR within the composition of the fourth aspect of the invention, has increased stability to any one of heat, a detergent, a chaotropic agent and an extreme of pH.

Preferably, the mutant GPCR has increased stability (e.g. thermostability) compared to its parent when in the presence or absence of a ligand thereto. Typically, the ligand is an antagonist, a full agonist, a partial agonist or an inverse agonist, whether orthosteric or allosteric. The ligand may be a polypeptide, such as an antibody.

It is preferred that the mutant GPCR of the second or third aspect of the invention, or the mutant GPCR within the composition of the fourth aspect of the invention, is at least 1° C. or 2° C. more stable than its parent, preferably at least 5° C. more stable, more preferably at least 8° C. more stable and even more preferably at least 10° C. or 15° C. or 20° C. more stable than its parent. Typically, thermostability of the parent and mutant receptors are measured under the same conditions. Typically, thermostability is assayed under a condition in which the GPCR resides in a particular conformation. Typically, this selected condition is the presence of a ligand which binds the GPCR.

It is preferred that the mutant GPCR of the second or third aspect of the invention, or the mutant GPCR within the composition of the fourth aspect of the invention, when solubilised and purified in a suitable detergent has a similar thermostability to bovine rhodopsin purified in dodecyl maltoside; however it is appreciated that any increase in stability will be useful for applications such as crystallisation studies. It is particularly preferred that the mutant GPCR of the second or third aspect of the invention, or the mutant GPCR within the composition of the fourth aspect of the invention retains at least 50% of its ligand binding activity after heating at 40° C. for 30 minutes. It is further preferred that the mutant GPCR of the second or third aspect of the invention, or the mutant GPCR within the composition of the fourth aspect of the invention retains at least 50% of its ligand binding activity after heating at 55° C. for 30 minutes.

The mutant GPCRs and compositions disclosed herein are useful for crystallisation studies and are useful in drug discovery programmes. They may be used in biophysical measurements of receptor/ligand kinetic and thermodynamic parameters eg by surface plasmon resonance or fluorescence based techniques. They may be used in ligand binding screens, and may be coupled to solid surfaces for use in high throughput screens or as biosensor chips. Biosensor chips containing the mutant GPCRs or compositions may be used to detect molecules, especially biomolecules.

The invention also includes a polynucleotide which encodes a mutant GPCR of the second or third aspect of the invention. In particular, polynucleotides are included which encode the mutant adenosine, mutant serotonin or mutant muscarinic receptors of the invention. The polynucleotide may be DNA or it may be RNA. Typically, it is comprised in a vector, such as a vector which can be used to express the said mutant GPCR. Suitable vectors are ones which propagate in and/or allow the expression in bacterial or mammalian or insect cells.

The invention also includes host cells, such as bacterial or eukaryotic cells, which contain a polynucleotide which encodes the mutant GPCR. Suitable cells include E. coli cells, yeast cells, mammalian cells and insect cells.

The listing or discussion of an apparently prior-published document in this specification should not necessarily be taken as an acknowledgement that the document is part of the state of the art or is common general knowledge.

The invention will now be described in more detail with respect to the following Figures and Examples wherein:

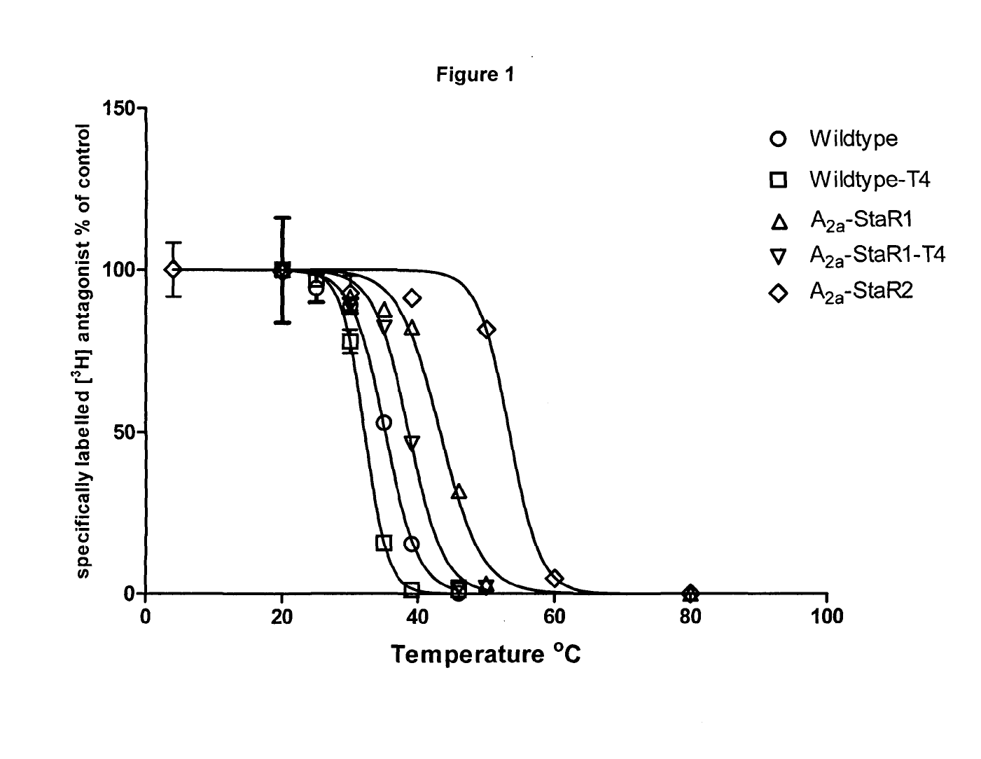

FIG. 1: Thermostability of the adenosine A_{2A} receptor alone, in combination with T4 lysozyme fusion or with thermostabilising mutations. Receptors were solubilised from transiently transfected cells using 0.025% DDM. Samples were heated at the specified temperature for 30 minutes, quenched on ice and the amount of receptor remaining was determined by a single-point binding assay using 100 nM [³H]-ZM-241,385. Data is shown for the wild type A₂ₐ receptor (circles), A_{2A}-T4L (squares), A_{2A}-StaR1 (previously Rant21, upright triangles), A_{2A}-T4 engineered to include the A_{2A}-StaR1 mutations in combination with the T4L fusion (A54L^{2.52}/T88A^{3.36}/K122A^{4.43} N239A^{6.41}) (inverted triangles) and vs A₂ₐ-StaR2 (diamonds).

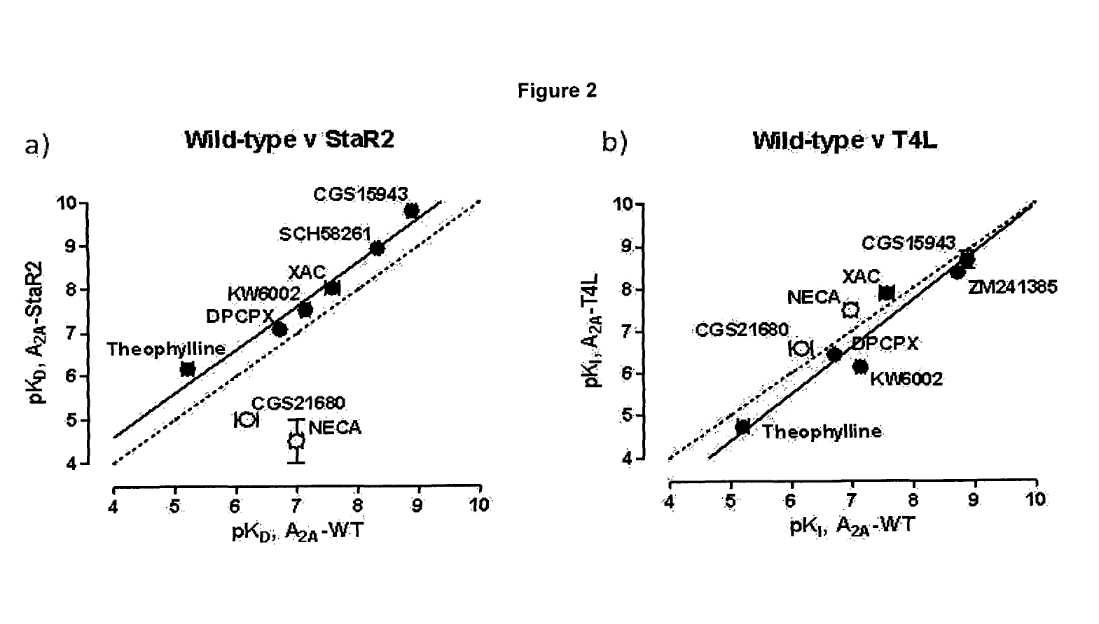

FIG. 2: Comparison of the pharmacology of A_{2A}-StaR2 and A_{2A}-T4 with the wild type A_{2A} receptor. Radioligand competition binding assays for a range of antagonists (closed circles) and agonists (open circles) were carried out with [³H]-ZM-241,385 on membranes from cells transiently transfected with receptors. a) Comparison of A_{2A}-StaR2 with wild type receptor and b) Comparison of A_{2A}-T4 with wild type receptor. pK_{D} values are listed in Table 1. Solid lines represent Deming regressions for antagonist affinities at wild-type and crystallographic constructs.

FIG. 3: Amino Acid sequence of human adenosine A_{2A} receptor (SEQ ID NO: 1).

FIG. 4: Alignment of Class 1 GPCR amino acid sequences (SEQ ID NOs: 2-98). Highlighted regions indicate positions of 3.55, 5.63 and 7.42 amino acid residues for each GPCR. The amino acid sequence of the adenosine A_{2A} receptor is underlined.

### EXAMPLE 1

### Thermostabilisation of the Adenosine A_{2A} Receptor

### Summary

G protein-coupled receptors play a key role in transducing extracellular signals to the cell interior and are known to function as dimers. Structural information on these receptors has been sparse and the mechanism of receptor dimerization unknown. Here we report the stabilisation of the adenosine A_{2A} receptor which has enabled us to solve the structure of the adenosine A_{2A} receptor in complex with the inverse agonist ZM-241,385, to reveal the features of an inactive state receptor.

### Introduction

The adenosine A_{2A} receptor is one of 4 GPCRs (A₁, A_{2A}, A_{2B}, A₃) activated by adenosine. Adenosine represents an important modulator of the central nervous system and periphery. In the brain adenosine controls neuronal excitability and the psychoactive effects of caffeine are mediated by adenosine receptors. A_{2A} receptors are located in the striatum and are considered a target for neurodegenerative disease ⁸. A_{2A} receptors are also expressed on the vasculature and immune cells where they have vasodilatory and anti-inflammatory effects ^{9 10}. There is growing evidence that drugs acting at adenosine receptors represent promising approaches in a wide range of diseases ¹⁰.

Mechanistic understanding of ligand binding and activation, as well as our ability to design drugs for GPCRs, including adenosine receptors, is hampered by the lack of structural information which largely stems from the instability of GPCRs outside of their native membrane environment. Here we report a general approach for stabilising GPCRs.

### Results and Discussion

### Thermostabilisation of the A_{2A} Receptor

To obtain a thermally stable receptor with a pre-defined conformation a technique of conformational stabilisation was employed, as previously used for the β1AR¹² and neurotensin receptor. Such thermostabilised receptors are known as StaRs for ‘stabilised receptors’ ¹³. The A_{2A} receptor was previously stabilised in both agonist and inverse agonist conformations¹⁴, however the stabilised inverse agonist receptor known as Rant21 or A_{2A}-StaR1 (containing the stabilising mutations A54L^{2.52}, T88A^{3.36}, K122A^{4.43} V239A^{6.41}; superscripts refer to Ballesteros-Weinstein numbering) was not considered of sufficient stability for structural studies. Further mutagenesis in the presence of the inverse agonist ligand ZM-241,385 20 resulted in the identification of an additional 4 stabilising mutations (R107A^{3.55}, L202A^{5.63}, L235A^{6.37}, S277A^{7.42}) giving an apparent thermostability of 47° C. in 0.1% decylmaltoside (FIG. 1) resulting in A_{2A}-StaR2. For crystallisation A_{2A}-StaR2 was truncated at the C-terminus by 96 amino acids up to Ala316 and included a C-terminal decameric His-tag for purification. An N154A mutation was introduced to remove the glycosylation site.

### Pharmacology of the Inverse Agonist State

The engineered receptor A_{2A}-StaR2 bound ZM-241,385 (K_{D} 1.9 nM) and a range of structurally diverse set of antagonists with a similar affinity to the wild type receptor¹³. In contrast, the affinities of agonists including NECA and CGS21860 were reduced by greater than 100-fold and the receptor no longer activated G proteins. This pharmacology is consistent with that expected for the inverse agonist conformation and is similar to the change in pharmacology observed for the stabilised β1AR-m23¹². This profile differs from A_{2A}-T4L which has a high agonist affinity¹¹ more consistent with the active conformation (FIG. 2). The thermostabilising residues Thr88^{3.36} and Ser277^{7.42} lie at the bottom of the predicted agonist binding pocket and have previously been shown to play a role in agonist binding and activation¹⁵⁻¹⁶. Mutation of these residues is highly stabilising to the antagonist but not the agonist conformation suggesting that they play a key role in the conformational selection of the receptor. The reduced agonist binding of A_{2A}-StaR2 is likely to be in part a direct effect of these mutations as well as the conformational stabilisation of the inverse agonist state as previously shown for β1AR-m23¹².

TABLE 1
Comparison of the pharmacology of A_{2A}-StaR2 and A_{2A}-T4
with the wild type A_{2A} receptor. Affinity values for a range of
agonist and antagonists from radioligand competition binding
assays using [³H]-ZM241,385 with membranes prepared
from HEK293 cells transiently transfected with receptors.

| | pK_{D}, wild-type | pK_{D}, StaR2 | pK_{D}, T4L |
|---|---|---|---|
| Theophylline | 5.2 ± 0.1 | 6.2 ± 0.0 | 4.7 ± 0.1 |
| XAC | 7.6 ± 0.1 | 8.0 ± 0.0 | 7.9 ± 0.0 |
| CGS15943 | 8.9 ± 0.1 | 9.8 ± 0.1 | 8.7 ± 0.3 |
| DPCPX | 6.7 ± 0.0 | 7.1 ± 0.0 | 6.4 ± 0.3 |
| SCH58261 | 8.3 ± 0.0 | 8.9 ± 0.0 | not tested |
| KW-6002 | 7.1 ± 0.1 | 7.5 ± 0.1 | 6.1 ± 0.1 |
| CGS21680 | 6.2 ± 0.2 | <5.0 | 6.6 ± 0.1 |
| NECA | 7.0 ± 0.1 | <5.0 | 7.5 ± 0.1 |

TABLE 2
Data of the individual stabilising mutations in A_{2A}-StaR1
showing the change in soluble expression and corresponding
shift in the thermostability.

| Mutation | % Wild type expression | Tm shift (° C.) |
|---|---|---|
| R107A | 8.9 | +2.1 |
| L202A | 85.1 | +7.1 |
| S277A | 111.7 | +3.8 |

### Methods

### Ligand-Binding and Thermostability Assays.

Transfected HEK293T cells were resuspended in ice cold buffer [50 mM Tris pH7.4; 400 mM NaCl; 1% DDM and protease inhibitors (Complete, Roche)]. After incubation for 1 h at 4° C., samples were centrifuged (16,000×g, 20 min, 4° C.) and the supernatant was detergent exchanged into 0.1% decylmaltoside (DM) from Ni-NTA resin.

Thermostability was assessed by incubating with [³H]-ZM241385 radioligand (100 nM) at increasing temperatures for 30 min followed by a 5 minutes incubation on ice. Receptor bound and free radioligand were separated by gel filtration as previously described'

### Membrane Radioligand Binding

Membranes from transfected HEK293 cells were incubated with [³H]-ZM241385 as previously described⁹ in the presence or absence competing compounds. After 90 min incubation at room temperature assays were terminated by rapid filtration and bound ligand measured by scintillation spectroscopy.

### REFERENCES

1. S. H. White (2004) Protein Sci 13, 1948-1949.
2. C. G. Tate (2001) FEBS Lett 504, 94-98.
3. R. Grisshammer, C. G. Tate (1995) Q Rev Biophys 28, 315-422.
4. J. U. Bowie (2001) Curr Opin Struct Biol 11, 397-402.
5. F. W. Lau, S, Nauli, Y. Zhou, J. U. Bowie (1999) J Mol Biol 290, 559-564.
6. Y. Zhou, J. U. Bowie (2000) J Biol Chem 275, 6975-6979.
7. S. Faham, D. Yang, E. Bare, S. Yohannan, J. P. Whitelegge, J. U. Bowie (2004) J Mol Biol 335, 297-305.
8. Simola, N., Morelli, M. & Pinna, A. Adenosine A2A receptor antagonists and Parkinson's disease: state of the art and future directions. Curr Pharm Des 14, 1475-1489 (2008).
9. Hasko, G., Linden, J., Cronstein, B. & Pacher, P. Adenosine receptors: therapeutic aspects for inflammatory and immune diseases. Nat Rev Drug Discov 7, 759-770, doi:nrd2638 [pii] 10.1038/nrd2638 (2008).
10. Jacobson, K. A. & Gao, Z. G. Adenosine receptors as therapeutic targets. Nat Rev Drug Discov 5, 247-264, doi:nrd1983 [pii] 10.1038/nrd1983 (2006).
11. Jaakola, V. P. et al. The 2.6 angstrom crystal structure of a human A₂ₙ adenosine receptor bound to an antagonist. Science 322, 1211-1217, doi:1164772 [pii] 10.1126/science.1164772 (2008).
12. Serrano-Vega, M. J., Magnani, F., Shibata, Y. & Tate, C. G. Conformational thermostabilization of the β₁-adrenergic receptor in a detergent-resistant form. Proc Natl Acad Sci USA 105, 877-882, doi:0711253105 [pii] 10.1073/pnas.0711253105 (2008).
13. Robertson, N. et al. The properties of thermostabilised G protein-coupled receptors (StaRs) and their use in Drug Discovery. Neuropharmacology, doi:S0028-3908(10)00162-0 [pii] 10.1016/j.neuropharm.2010.07.001 (2010).
14. Magnani, F., Shibata, Y., Serrano-Vega, M. J. & Tate, C. G. Co-evolving stability and conformational homogeneity of the human adenosine A₂ₐ receptor. Proc Natl Acad Sci USA 105, 10744-10749, doi:0804396105 [pii] 10.1073/pnas.0804396105 (2008).
15. Ivanov, A. A., Barak, D. & Jacobson, K. A. Evaluation of homology modeling of G-protein-coupled receptors in light of the A(2A) adenosine receptor crystallographic structure. J Med Chem 52, 3284-3292, doi:10.1021/jm801533x (2009).
16. Jiang, Q. et al. Hydrophilic side chains in the third and seventh transmembrane helical domains of human A_{2A}n adenosine receptors are required for ligand recognition. Mol Pharmacol 50, 512-521 (1996).

1. A method for producing a mutant G-protein coupled receptor (GPCR) with increased stability relative to a parent GPCR, the method comprising making one or more mutations in the amino acid sequence that defines a Class 1 parent GPCR, wherein (i) the one or more mutations are located within a window of i plus or minus 5 residues, where i is the position of amino acid residue 3.55 in the parent GPCR, and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, to provide one or more mutants of the parent GPCR with increased stability.

2. A method according to claim 1, wherein the one or more mutants of the parent GPCR have increased stability of a particular conformation.

3. A method according to claim 2, wherein the one or more mutants have increased stability in an agonist or antagonist conformation.

4. A method according to claim 1, wherein the one or more mutants of the parent GPCR have increased stability to any one or more of heat, a detergent, a chaotropic agent and an extreme of pH.

5. A method according to claim 4 wherein the one or more mutants have increased thermostability.

6. (canceled)

7. A method according to claim 1, wherein the parent GPCR is any of an adenosine receptor, a serotonin receptor, a β-adrenergic receptor, a neurotensin receptor, a muscarinic receptor, or an orexin receptor.

8. A method according to claim 1, wherein the mutant GPCR with increased stability relative to its parent GPCR, is a mutant adenosine receptor which, when compared to the corresponding parent receptor, has a different amino acid at a position which corresponds to any one or more of Arginine 107, Leucine 202 and Serine 277 according to the numbering of the human adenosine A_{2A} receptor as set out in SEQ ID NO:1.

9. A method according to claim 8 wherein the mutant adenosine receptor is a mutant adenosine A_{2A}, A_{2B}, A₃ or A₁ receptor.

10. (canceled)

11. A mutant GPCR with increased stability relative to its parent GPCR produced by the method of claim 1.

12. A mutant GPCR which, when compared to a Class 1 parent GPCR, has one or more mutations in the amino acid sequence defining the parent GPCR, wherein (i) the one or more mutations are located within a window of i plus or minus 5 residues, where i is the position of amino acid residue 3.55 in the parent GPCR, and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, which mutant GPCR has increased stability compared to a parent GPCR when exposed to a destabilising condition.

13. A composition comprising a mutant GPCR which, when compared to a Class 1 parent GPCR, has one or more mutations in the amino acid sequence defining the parent GPCR, wherein (i) the one or more mutations are located within a window of i plus or minus 5 residues, where i is the position of amino acid residue 3.55 in the parent GPCR, and/or (ii) the one or more mutations are located within a window of i minus 2 to i residues, where i is the position of amino acid residue 5.63 in the parent GPCR, and/or (iii) the one or more mutations are located within a window of i minus 4 to i plus 1 residues, where i is the position of amino acid residue 7.42 in the parent GPCR, characterised in that the mutant GPCR is exposed to a destabilising condition effective to destabilise a parent GPCR to a greater extent than the mutant GPCR.

14. A mutant GPCR according to claim 12, wherein the mutant GPCR is a mutant adenosine receptor which, when compared to the corresponding parent receptor, has a different amino acid at a position which corresponds to any one or more of Arginine 107, Leucine 202 and Serine 277 according to the numbering of the human adenosine A_{2A} receptor as set out in SEQ ID NO:1.

15. (canceled)

16. A mutant GPCR according to claim 12, wherein the mutant GPCR is any of a mutant adenosine A_{2B}, A₃ or A₁ receptor, a mutant serotonin receptor, a mutant β-adrenergic receptor, a mutant neurotensin receptor, a mutant muscarinic acid receptor, a mutant orexin receptor, a mutant 5-hydroxytryptamine receptor, a mutant adrenoceptor, a mutant anaphylatoxin receptor, a mutant angiotensin receptor, a mutant apelin receptor, a mutant bombesin receptor, a mutant bradykinin receptor, a mutant chemokine receptor, a mutant cholecystokinin receptor, a mutant dopamine receptor, a mutant endothelin receptor a mutant free fatty acid receptor, a mutant bile acid receptor, a mutant galanin receptor, a mutant motilin receptor, a mutant ghrelin receptor, a mutant glycoprotein hormone receptor, a mutant GnRH receptor, a mutant histamine receptor, a mutant KiSS1-derived peptide receptor, a mutant leukotriene and lipoxin receptor, a mutant lysophospholipid receptor, a mutant melanin-concentrating hormone receptor, a mutant melanocortin receptor, a mutant melatonin receptor, a mutant neuromedin U receptor, a mutant neuropeptide receptor, a mutant N-formylpeptide family receptor, a mutant nicotinic acid receptor, a mutant opiod receptor, a mutant op sin-like receptor, a mutant P2Y receptor, a mutant peptide P518 receptor, a mutant platelet-activating factor receptor, a mutant prokineticin receptor, a mutant prolactin-releasing peptide receptor, a mutant pro stanoid receptor, a mutant protease-activated receptor, a mutant relaxin receptor, a mutant somatostatin receptor, a mutant SPC/LPC receptor, a mutant tachykinin receptor, a mutant trace amino receptor, a mutant thryotropin-releasing hormone receptor, a mutant urotensin receptor, a mutant vasopressin/oxytocin receptor, a mutant orphan GPCR, or a mutant cannabinoid receptor.

17. A mutant GPCR according to claim 12, wherein (i) when the mutant GPCR is a mutant adenosine A_{2A} receptor, it does not have, when compared to the corresponding parent receptor, a different amino acid at a position which corresponds to Serine 277 according to the numbering of the human adenosine A_{2A} receptor as set out in SEQ ID NO:1, or wherein (ii) when the mutant GPCR is a mutant human adenosine A_{2A} receptor, it does not have an alanine amino acid at each of the positions corresponding to Serine 277, Arginine 107 and Leucine 202 according to the numbering of the human adenosine A_{2A} receptor as set out in SEQ ID NO:1.

18. (canceled)

19. A mutant GPCR according to claim 12, wherein the mutant GPCR is membrane free.

20. A mutant GPCR according to claim 12, wherein the mutant GPCR has increased stability compared to a parent GPCR in the absence of a ligand.

21. A mutant GPCR according to claim 12, wherein the mutant GPCR has increased stability compared to its parent GPCR when in the presence of a ligand.

22. (canceled)

23. A mutant GPCR according to claim 12, wherein the mutant GPCR has increased stability to any one of heat, a detergent, a chaotropic agent and an extreme of pH.

24. A mutant GPCR according to claim 12, wherein the mutant GPCR has increased thermo stability.

25. (canceled)

26. A mutant GPCR according to claim 12, wherein the mutant GPCR is in a solubilised form.

27-33. (canceled)


SEQUENCE LISTING (98 sequences)

SEQ ID NO: 1 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 2 - protein, 235 aa, Artificial Sequence
  Consensus sequence of Class 1 GPCR amino acid sequences
SPWQAVLLAALYSLVFLLGLLGNVLVILVILRNKTVTNYFLLNLAVADLLVALTLPPWAAYYALGGGRVGCKLWLALDVLNCTASILSLTAISVDRYLAIVHTPRRAKVVIAAVWVLSLLISLPPLLFWNVAYVIYSFILGFYLPLLIMLVCYGRIYRTRKATKMLVIVVGVFVLCWLPFFIVNLLNAFCPGSIIFQVTIWLGYANSCLNPIIYAFLNKRKAFLKLLCCRCRSSP

SEQ ID NO: 3 - protein, 244 aa, Homo sapiens
VNISKAILLGVILGGLILFGVLGNILVILSVACHRHSVTHYYIVNLAVADLLLTSTVLPFSAIFEVLGFGRVFCNIWAAVDVLCCTASIMGLCIISIDRYIGVSYVTQRRGLMALLCVWALSLVISIGPLFGNEEPGYVLFSALGSFYLPLAIILVMYCRVYVVEKKAAKTLGIVVGCFVLCWLPFFLVMPIGSFFPDFKPVFKIVFWLGYLNSCINPIIYPCSSQARVRSKSFLQVCCCVGPS

SEQ ID NO: 4 - protein, 245 aa, Homo sapiens
LDITRAISVGLVLGAFILFAIVGNILVILSVACNRRTPTNYFIVNLAMADLLLSFTVLPFSAALEVLGLGRIFCDIWAAVDVLCCTASILSLCAISIDRYIGVRYVTRRKAILALLSVWVLSTVISIGPLLGTEEPFYALFSSLGSFYIPLAVILVMYCRVYIVEKKAAKTLGIVVGMFILCWLPFFIALPLGSLFSTLKPVFKVVFWLGYFNSCLNPIIYPCSSKEFKRAFVRILGCQCRGRRR

SEQ ID NO: 5 - protein, 244 aa, Homo sapiens
VVSAQGVGVGVFLAAFILMAVAGNLLVILSVACNRQTVTNYFIVNLAVADLLLSATVLPFSATMEVLGFGRAFCDVWAAVDVLCCTASILSLCTISVDRYVGVRHMTERKAAAILALLWVVALVVSVGPLLGTEEAGYAVFSSVCSFYLPMAVIVVMYCRVYVVEKKAAKTLAIVVGVFVLCWFPFFFVLPLGSLFPQLKPVFKVIFWLGYFNSCVNPLIYPCSSRFRRPTTQLRAKVSSLSHK

SEQ ID NO: 6 - protein, 246 aa, Homo sapiens
YSLQVTLTLVCLAGLLMLLTVFGNVLVIIAVFTSRKAPQNLFLVSLASADILVATLVIPFSLANEVMGFGKAWCEIYLALDVLFCTSSIVHLCAISLDRYWSITQRTPRRIKAIIITVWVISAVISFPPLISNDQKWYVISSCIGSFFAPCLIMILVYVRIYQIEKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 7 - protein, 245 aa, Homo sapiens
YSVQATAAIAAAITFLILFTIFGNALVILAVLTSRRAPQNLFLVSLAAADILVATLIIPFSLANELLGFRRTWCEVYLALDVLFCTSSIVHLCAISLDRYWAVSRRTPRRIKCIILTVWLIAAVISLPPLIYNQEAWYILASSIGSFFAPCLIMILVYLRIYLIEKRFTFVLAVVIGVFVLCWFPFFFSYSLGAICPKHCKLFQFFFWIGYCNSSLNPVIYTIFNQDFRRAFRRILCRPWTQTAW

SEQ ID NO: 8 - protein, 245 aa, Homo sapiens
YSAGAVAGLAAVVGFLIVFTVVGNVLVVIAVLTSRRAPQNLFLVSLASADILVATLVMPFSLANELMAFGQVWCGVYLALDVLFCTSSIVHLCAISLDRYWSVTQRTPRRVKATIVAVWLISAVISFPPLVSNDETWYILSSCIGSFFAPCLIMGLVYARIYRVEKRFTFVLAVVMGVFVLCWFPFFFIYSLYGICREACQLFKFFFWIGYCNSSLNPVIYTVFNQDFRPSFKHILFRRRRRGFR

SEQ ID NO: 9 - protein, 245 aa, Homo sapiens
YSAGAVAGLAAVVGFLIVFTVVGNVLVVIAVLTSRRAPQNLFLVSLASADILVATLVMPFSLANELMAFGQVWCGVYLALDVLFCTSSIVHLCAISLDRYWSVTQRTPRRVKATIVAVWLISAVISFPPLVSNDETWYILSSCIGSFFAPCLIMGLVYARIYRVEKRFTFVLAVVMGVFVLCWFPFFFSYSLYGICREACQLFKFFFWIGYCNSSLNPVIYTVFNQDFRRSFKHILFRRRRRGFR

SEQ ID NO: 10 - protein, 244 aa, Homo sapiens
LSQQWTAGMGLLMALIVLLIVAGNVLVIVAIAKTPQTLTNLFIMSLASADLVMGLLVVPFGATIVVWGYGSFFCELWTSVDVLCVTASIETLCVIALDRYLAITSLTRARARGLVCTVWAISALVSFLPILMVTNRAYAIASSVVSFYVPLCIMAFVYLRVFREEQKALKTLGIIMGVFTLCWLPFFLANVVKAFHRELVPLFVFFNWLGYANSAFNPIIYCRSPDFRKAFQGLLCCARRAARR

SEQ ID NO: 11 - protein, 244 aa, Homo sapiens
RDEVWVVGMGIVMSLIVLAIVFGNVLVITAIAKFEQTVTNYFITSLACADLVMGLAVVPFGAAHILMKFGNFWCEFWTSIDVLCVTASIETLCVIAVDRYFAITSLTKNKARVIILMVWIVSGLTSFLPIQMFTNQAYAIASSIVSFYVPLVIMVFVYSRVFQEEHKALKTLGIIMGTFTLCWLPFFIVNIVHVIQDNLIRVYILLNWIGYVNSGFNPLIYCRSPDFRIAFQELLCLRRSSLKA

SEQ ID NO: 12 - protein, 244 aa, Homo sapiens
GVPWEAALAGALLALAVLATVGGNLLVIVAIAWTPQTMTNVFVTSLAAADLVMGLLVVPPAATLALTGLGATGCELWTSVDVLCVTASIETLCALAVDRYLAVTNVTKRCARTAVVLVWVVSAAVSFAPIMSASNMPYVLLSSSVSFYLPLLVMLFVYARVFVVEHRALCTLGLIMGTFTLCWLPFFLANVLRALGGPSLVAFLALNWLGYANSAFNPLIYCRSPDFRSAFRRLLCRCGRRLPP

SEQ ID NO: 13 - protein, 246 aa, Homo sapiens
SISAFQAAYIGIEVLIALVSVPGNVLVIWAVKVNQRDATFCFIVSLAVADVAVGALVIPLAILINIGPQTYFHTCLMVACPVLILTQSSILALLAIAVDRYLRVKIVTPRRAAVAIAGCWILSFVVGLTPMFGISMEYMVYFNFFVWVLPPLLLMVLIYLEVFYLELKIAKSLALILFLFALSWLPLHILNCITLFCPSCHKLTYIAIFLTHGNSAMNPIVYAFRIQKFRVTFLKIWNDHFRCQPA

SEQ ID NO: 14 - protein, 246 aa, Homo sapiens
MPIMGSSVYITVELAIAVLAILGNVLVCWAVWLNSQNVTNYFVVSLAAADIAVGVLAIPFAITISTGFCAACHGCLFIACFVLVLTQSSIFSLLAIAIDRYIAIRIVTGTRAKGIIAICWVLSFAIGLTPMLGVPMNYMVYFNFFACVLVPLLLMLGVYLRIFLAEVHAAKSLAIIVGLFALCWLPLHIINCFTFFCPDCSHLMYLAIVLSHTNSVVNPFIYAYRIREFRQTFRKIIRSHVLRQQE

SEQ ID NO: 15 - protein, 246 aa, Homo sapiens
LLETQDALYVALELVIAALSVAGNVLVCAAVGTANQTPTNYFLVSLAAADVAVGLFAIPFAITISLGFCTDFYGCLFLACFVLVLTQSSIFSLLAVAVDRYLAICVVTGTRARGVIAVLWVLAFGIGLTPFLGVPMSYMVYFNFFGCVLPPLLIMLVIYIKIFLVEIHAAKSLAMIVGIFALCWLPVHAVNCVTLFQPAQGKAMNMAILLSHANSVVNPIVYAYRNRDFRYTFHKIISRYLLCQAD

SEQ ID NO: 16 - protein, 247 aa, Homo sapiens
ALSLANVTYITMEIFIGLCAIVGNVLVICVVKLNPQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVMRMDYMVYFSFLTWIFIPLVVMCAIYLDIFYIEFKTAKSLFLVLFLFALSWLPLSIINCIIYFNGEVPQLVLYMGILLSHANSMMNPIVYAYKIKKFKETYLLILKACVVCHPS

SEQ ID NO: 17 - protein, 244 aa, Homo sapiens
NEDFKYVLLPVSYGVVCVLGLCLNAVALYIFLCRLWNASTTYMFHLAVSDALYAASLPLLVYYYARGDFSTVLCKLVRFLFYTNLYCSILFLTCISVHRCLGVLRGRARYARRVAGAVWVLVLACQAPVLYFSRFVAYSSVMLGLLFAVPFAVILVCYVLMARRKRKSVRTIAVVLAVFALCFLPFHVTRTLYYSFRSLDLAYKVTRPLASANSCLDPVLYFLAGQFRRTESTPAGSENTKDIR

SEQ ID NO: 18 - protein, 245 aa, Homo sapiens
DEDFKFILLPVSYAVVFVLGLGLNAPTLWLFIFRLWDATATYMFHLALSDTLYVLSLPTLIYYYAAHNFGTEICKFVRFLFYWNLYCSVLFLTCISVHRYLGICHGRPRLAGLLCLAVWLVVAGCLVPNLFFDHYVHFSSAVMGLLFGVPCLVTLVCYGLMARRRLRSLRTIAVVLTVFAVCFVPFHITRTIYYLARLLEAVYKVTRPLASANSCLDPVLYLLTGDKYRRQLRQLCGGGKPQPRT

SEQ ID NO: 19 - protein, 244 aa, Homo sapiens
NDSFKYTLYGCMFSMVFVLGLVSNCVAIYIFICVLRNETTTYMINLAMSDLLFVFTLPFRIFYFTTRNFGDLLCKISVMLFYTNMYGSILFLTCISVDRFLAIVYRTKRNAKIVCTGVWLTVIGGSAPAVFVTYLSRIVIFIEIVGFFIPLILNVTCSSMVLKTKTKVLKMIFVHLIIFCFCFVPYNINLILYSLVRTQTFMYPITLCIAVSNCCFDPIVYYFTSDFRFSEVHGAENFIQHNLQ

SEQ ID NO: 20 - protein, 246 aa, Homo sapiens
RENFKQLLLPPVYSAVLAAGLPLNICVITQICTSRLTRTAVYTLNLALADLLYACSLPLLIYNYAQGDFGDFACRLVRFLFYANLHGSILFLTCISFQRYLGICHPLAPWHKRGGRRAAWLVCVAVWLAVTTQTHYMPYGMALTVIGFLLPFAALLACYCLLACRRGKAARMAVVVAAAFAISFLPFHITKTAYLAVRSTPGAYKGTRPFASANSVLDPILFYFTQKKFRRRPHELLQKLTAKWQR

SEQ ID NO: 21 - protein, 244 aa, Homo sapiens
GVEFISLLAIILLSVALAVGLPGNSFVVWSILKRMRSVTALMVLNLALADLAVLLTAPFFLHFLAQGTFGLAGCRLCHYVCGVSMYASVLLITAMSLDRSLAVARRTKAMARRVLAGIWVLSFLLATPVLAYGHRAFHLIFEAVTGFLLPFLAVVASYSDIGRRSRRTGRLVVLIILTFAAFWLPYHVVNLAEAGRALAGQARNVLIALAFLSSSVNPVLYACAGGLRSAGVGFVAKLLEGTGS

SEQ ID NO: 22 - protein, 244 aa, Homo sapiens
DDSFKYNLNGAVYSVVFILGLITNSVSLFVFCFRMRSETAIFITNLAVSDLLFVCTLPFKIFYNFNRHFGDTLCKISGTAFLTNIYGSMLFLTCISVDRFLAIVYRTRRNSAIVCAGVWILVLSGGISASLFTYLSKITIFIEVVGFIIPLILNVSCSSVVLRTKKKVLKMITVHMAVFVVCFVPYNSVLFLYALVRSQAIMYPITLCLATLNCCFDPFIYYFTLEIRMESLFKTETPLTTKPS

SEQ ID NO: 23 - protein, 245 aa, Homo sapiens
MTGFQFYYLPAVYILVFIIGFLGNSVAIWMFVFHMWSGISVYMFNLALADFLYVLTLPALIFYYFNKTFGDAMCKLQRFIFHVNLYGSILFLTCISAHRYSGVVYLKKKNAICISVLVWLIVVVAISPILFYRSYFIYSMCTTVAMFCVPLVLILGCYGLIVRARRKSIYLVIIVLTVFAVSYIPFHVMKTMNLRARLDFQTYQVTRGLASLNSCVDPILYFLAGDTFRRRLSRATRKASRRSEA

SEQ ID NO: 24 - protein, 244 aa, Homo sapiens
MGPWQVAFIGITTGLLSLATVTGNLLVLISFKVNTKTVNNYFLLSLACADLIIGTFSMNLYTTYLLMGLGTLACDLWLALDYVASNASVMNLLLISFDRYFSVTRRTPRRAALMIGLAWLVSFVLWAPAILFLSQPIITFGTAMAAFYLPVTVMCTLYWRIYREEKKAARTLSAILLAFILTWTPYNIMVLVSTFCKDCVPLWELGYWLCYVNSTINPMCYALCNKAFRDTFRLLLLCRWDKRR

SEQ ID NO: 25 - protein, 245 aa, Homo sapiens
YKTFEVVFIVLVAGSLSLVTIIGNILVMVSIKVNRQTVNNYFLFSLACADLIIGVFSMNLYTLYTVIGLGPVVCDLWLALDYVVSNASVMNLLIISFDRYFCVTKRTTKMAGMMIAAAWVLSFILWAPAILFFSNAAVTFGTAIAAFYLPVIIMTVLYWHISRAEKKVTRTILAILLAFIITWAPYNVMVLINTFCAPCIPVWTIGYWLCYINSTINPACYALCNATFKKTFKHLLMCHYKNIGA

SEQ ID NO: 26 - protein, 245 aa, Homo sapiens
MTVWQVVFIAFLTGILALVTIIGNILVIVSFKVNKKTVNNYFLLSLACADLIIGVISMNLFTTYIIMNLGNLACDLWLAIDYVASNASVMNLLVISFDRYFSITRRTTKRAGVMIGLAWVISFVLWAPAILFLSEPTITFGTAIAAFYMPVTIMTILYWRIYKEEKKAAQTLSAILLAFIITWTPYNIMVLVNTFCDSCIPFWNLGYWLCYINSTVNPVCYALCNKTFRTTFKMLLLCQCDKKKR

SEQ ID NO: 27 - protein, 242 aa, Homo sapiens
YETVEMVFIATVTGSLSLVTVVGNILVMLSIKVNRQTVNNYFLFSLACADLIIGAFSMNLYTVYIIKGLGAVVCDLWLALDYVVSNASVMNLLIISFDRYFCVTKRTTKMAGLMIAAAWVLSFVLWAPAILFLSNPAVTFGTAIAAFYLPVVIMTVLYIHISLAERKVTRTIFAILLAFILTWTPYNVMVLVNTFCQSCIPVWSIGYWLCYVNSTINPACYALCNAFRHLLLCQYRNIGTAR

SEQ ID NO: 28 - protein, 245 aa, Homo sapiens
MRLWEVITIAAVTAVVSLITIVGNVLVMISFKVNSKTVNNYYLLSLACADLIIGIFSMNLYTTYILMGLGSLACDLWLALDYVASNASVMNLLVISFDRYFSITRRTPKRAGIMIGLAWLISFILWAPAILCLSEPTITFGTAIAAFYIPVSVMTILYCRIYREERKAAQTLSAILLAFIITWTPYNIMVLVSTFCDKCVPLWHLGYWLCYVNSTVNPICYALCNRTFRKTFKMLLLCRWKKKKV

SEQ ID NO: 29 - protein, 239 aa, Homo sapiens
SDCPRVVLPEEIFFTISIVGVLENLIVLLAVFKNKQAPMYFFICSLAISDMLGSLYKILENILIILRNFETTADDIIDSLFVLSLLGSIFSLSVIAADRYITIFHVTMRRTVVVLTVIWTFCTGTGITMVIFSHHVPTVITFTSLFPLMLVFILCLYVHMFNMKGAITLTILLGVFIFCWAPFVLHVLLMTFCPSNPYLFQVNGMLIMCNAVIDPFIYAFRSPELRDAFKKMIFCSRYW

SEQ ID NO: 30 - protein, 244 aa, Homo sapiens
PSDKHLDAIPILYYIIFVIGFLVNIVVVTLFCCQKKKVSSIYIFNLAVADLLLLATLPLWATYYSYRYFGPVMCKVFGSFLTLNMFASIFFITCMSVDRYQSVIYRNPWQASYIVPLVWCMACLSSLPTFYFQWSAGIALMKNILGFIIPLIFIATCYFGIRKHRDQVLKMAAAVVLAFIICWLPFHVLTFLDALAWMGVIALPFAILLGFTNSCVNPFLYCFVGNFRVPITWLQGKRESMSCR

SEQ ID NO: 31 - protein, 245 aa, Homo sapiens
RHNYIFVMIPTLYSIIFVVGIFGNSLVVIVIYFYMKTVASVFLLNLALADLCFLLTLPLWAVYTAMEYFGNYLCKIASASVSFNLYASVFLLTCLSIDRYLAIVHRTMLVAKVTCIIIWLLAGLASLPAIIHTLPIGLGLTKNILGFLFPFLIILTSYTLIWKANDDIFKIIMAIVLFFFFSWIPHQIFTFLDVLIQLGIIAMPITICIAYFNNCLNPLFYGFLGKKFKRYFLQLLKYIPPKAKS

SEQ ID NO: 32 - protein, 245 aa, Homo sapiens
RHNYIFVMIPTLYSIIFVVGIFGNSLVVIVIYFYMKTVASVFLLNLALADLCFLLTLPLWAVYTAMEYFGNYLCKIASASVSFNLYASVFLLTCLSIDRYLAIVHRTMLVAKVTCIIIWLLAGLASLPAIIHTLPIGLGLTKNILGSCFPFLIILTSYTLIWKANDDIFRIIMAIVLFFFFSWIPHQIFTFLDVLIQQGIIAMPITIWIAYFNNCLNPLFYGFLGKKFKKDILQLLKYIPPKAKS

SEQ ID NO: 33 - protein, 245 aa, Homo sapiens
AWDLLHRVLPTFIISICFFGLLGNLFVLLVFLLPRLNVAEIYLANLAASDLVFVLGLPFWAENIWNQFFGALLCRVINGVIKANLFISIFLVVAISQDRYRVLVHQRRRQARVTCVLIWVVGGLLSIPTFLLAWHFARIVELNILGFLLPLAAIVFFNYHILASDSKTTALILTLVVAFLVCWAPYHFFAFLEFLFQVQAVGLQLANFFAFTNSSLNPVIYVFVGRLFRTKVWELYKQCTPKSLA

SEQ ID NO: 34 - protein, 245 aa, Homo sapiens
MLGWLNTIQPPFLWVLFVLATLENIFVLSVFCLHKCTVAEIYLGNLAAADLILACGLPFWAITISNNFFGETLCRVVNAIISMNLYSSICFLMLVSIDRYLALVKRGVRWAKLYSLVIWGCTLLLSSPMLVFIWEVFTNMLLNVVGFLLPLSVITFCTMQIMQVERRATVLVLVVLLLFIICWLPFQISTFLDTLHRLGILITQIASFMAYSNSCLNPLVYVIVGKRFRKKSWEVYQGVCQKGGC

SEQ ID NO: 35 - protein, 243 aa, Homo sapiens
VTVSYQVITSLLLGTLIFCAVLGNACVVAAIALERQNVANYLIGSLAVTDLMVSVLVLPMAALYQVLNLGQVTCDLFIALDVLCCTSSILHLCAIALDRYWAITDRTPRRAAALISLTWLIGFLISIPPMLGSKDHGYTIYSTFGAFYIPLLLMLVLYGRIFRAERKTVKTLGIIMGTFILCWLPFFIVALVLPFCESSCHLGAIINWLGYSNSLLNPVIYAYFNKDFQNAFKKIIKCKFCRQ

SEQ ID NO: 36 - protein, 242 aa, Homo sapiens
ISLPWKVLLVMLLALITLATTLSNAFVIATVYRTRHTPANYLIASLAVTDLLVSILVMPISTMYTVTGLGQVVCDFWLSSDITCCTASILHLCVIALDRYWAITDRTPKRAAVMIALVWVFSISISLPPFFWTDHILYTVYSTVGAFYFPTLLLIALYGRIYVEERKATKTLGIILGAFIVCWLPFFIISLVMPICKDACWIFDFFTWLGYLNSLINPIIYTMSNEDFKQAFHKLIRFKCTS

SEQ ID NO: 37 - protein, 242 aa, Homo sapiens
TLQALKISLAVVLSVITLATVLSNAFVLTTILLTRHTPANYLIGSLATTDLLVSILVMPISIAYTITHFGQILCDIWLSSDITCCTASILHLCVIALDRYWAITDRTAGHAATMIAIVWAISICISIPPLFWTSQISYTIYSTCGAFYIPSVLLIILYGRIYRAERKATKILGIILGAFIICWLPFFVVSLVLPICRDSCWLFDFFTWLGYLNSLINPIIYTVFNEEFRQAFQKIVPFRKAS

SEQ ID NO: 38 - protein, 242 aa, Homo sapiens
MTITEKMLICMTLVVITTLTTLLNLAVIMAIGTTKHQPANYLICSLAVTDLLVAVLVMPLSIIYIVMDLGYFLCEVWLSVDMTCCTCSILHLCVIALDRYWAITNRTAKRAALMILTVWTISIFISMPPLFWHDHVIYTIYSTLGAFYIPLTLILILYYRIYHAERKAARILGLILGAFILSWLPFFIKELIVGLSIYTVSVADFLTWLGYVNSLINPLLYTSFNEDFKLAFKKLIRCREHT

SEQ ID NO: 39 - protein, 240 aa, Homo sapiens
NRMPSKILVSLTLSGLALMTTTINSLVIAAIIVTRHHPANYLICSLAVTDFLVAVLVMPFSIVYIVREMGQVVCDIWLSVDITCCTCSILHLSAIALDRYRAITDRTPKHAGIMITIVWIISVFISMPPLFWHDHIVSTIYSTFGAFYIPLALILILYYKIYRAERKAATTLGLILGAFVICWLPFFVKELVVNVCDKCKIMSNFLAWLGYLNSLINPLIYTIFNEDFKKAFQKLVRCRC

SEQ ID NO: 40 - protein, 245 aa, Homo sapiens
LHLQEKNWSALLTAVVIILTIAGNILVIMAVSLEKQNATNYFLMSLAIADMLLGFLVMPVSMLTILYGLPSKLCAVWIYLDVLFSTASIMHLCAISLDRYVAIQNNSRTKAFLKIIAVWTISVGISMPIPVFLADDNFVLIGSFVSFFIPLTIMVITYFLTIKSEQKACKVLGIVFFLFVVMWCPFFITNIMAVICKESCNLLNVFVWIGYLSSAVNPLVYTLFNKTYRSAFSRYIQCQYKENKK

SEQ ID NO: 41 - protein, 245 aa, Homo sapiens
EQGNKLHWAALLILMVIIPTIGGNTLVILAVSLEKQYATNYFLMSLAVADLLVGLFVMPIALLTIMFELPLVLCPAWLFLDVLFSTASIMHLCAISVDRYIAIKKNSRATAFIKITVVWLISIGIAIPVPIKERFGDFMLFGSLAAFFTPLAIMIVTYFLTIHAEQRASKVLGIVFFLFLLMWCPFFITNITLVLCDSCNQLLEIFVWIGYVSSGVNPLVYTLFNKTFRDAFGRYITCNYRATKS

SEQ ID NO: 42 - protein, 245 aa, Homo sapiens
MPDGVQNWPALSIVIIIIMTIGGNILVIMAVSMEKHNATNYFLMSLAIADMLVGLLVMPLSLLAILYDLPRYLCPVWISLDVLFSTASIMHLCAISLDRYVAIRNNSRTKAIMKIAIVWAISIGVSVPIPVILNDPNFVLIGSFVAFFIPLTIMVITYCLTIYVERKASKVLGIVFFVFLIMWCPFFITNILSVLCEKSCNLLNVFVWIGYVCSGINPLVYTLFNKIYRRAFSNYLRCNYKVEKK

SEQ ID NO: 43 - protein, 208 aa, Homo sapiens
MDLPVNLTSFSLSTPSPLETNHSLGKDDLRPSSPLLSVFGVLILTLLGRRLCQLWIACDVLCCTASIWNVTAIALDRYWSITRTRKCVSNVMIALTWALSAVISLAPLLFSREPSYAVFSTVGAFYLPLCVVLFVYWKIYKAEQRAALMVGILIGVFVLCWIPFFLTELISPLCSCDIPWKSIFLWLGYSNSFFNPLIYTAFNKSRQH

SEQ ID NO: 44 - protein, 245 aa, Homo sapiens
SAPGGSGWVAAALCVVIALTAAANSLLIALICTQPRNTSNFFLVSLFTSDLMVGLVVMPPAMLNALYGLARGLCLLWTAFDVMCCSASILNLCLISLDRYLLILSMTPLRALALVLGAWSLAALASFLPLLLLASLPFVLVASGLTFFLPSGAICFTYCRILLAALKASLTLGILLGMFFVTWLPFFVANIVQAVCDCISPGLFDVLTWLGYCNSTMNPIIYPLFMRLRLTAQLLLPGEATQDPP

SEQ ID NO: 45 - protein, 245 aa, Homo sapiens
YGRVEKVVIGSILTLITLLTIAGNCLVVISVCFVKRQPSNYLIVSLALADLSVAVAVMPFVSVTDLIGFGHFFCNVFIAMDVMCCTASIMTLCVISIDRYLGITRQNGKCMAKMILSVWLLSASITLPPLFGSQDFGYTIYSTAVAFYIPMSVMLFMYYQIYKAEQKAATTLGIIVGAFTVCWLPFFLLSTARPFICGTSCVERTFLWLGYANSLINPFIYAFFNRDLRTTYRSLLQCQYRNINR

SEQ ID NO: 46 - protein, 246 aa, Homo sapiens
MNEPPVILSMVILSLTFLLGLPGNGLVLWVAGLKMQRTVNTIWFLHLTLADLLCCLSLPFSLAHLALQGYGRFLCKLIPSIIVLNMFASVFLLTAISLDRCLVVFKRNVGMACSICGCIWVVAFVMCIPVFVYTPLVAITITRLVVGFLLPSVIMIACYSFIVFRQSKTFRVAVVVVAVFLVCWTPYHIFGVLSLLTDPETPWDHVCIALASANSCFNPFLYALLGKDFRKKARQSIQGILEAAFS

SEQ ID NO: 47 - protein, 246 aa, Homo sapiens
IVVFGTVFLSIFYSVIFAIGLVGNLLVVFALTNSKKSVTDIYLLNLALSDLLFVATLPFWTHYLINEKGLHNAMCKFTTAFFFIGFFGSIFFITVISIDRYLAIVLRTVQHGVTISLGVWAAAILVAAPQFMFIWPVLRNVETNFLGFLLPLLIMSYCYFRIIQTKAKAIKLILLVVIVFFLFWTPYNVMIFLETLKLYDFFALSVTETVAFSHCCLNPLIYAFAGEKFRRYLYHLYGKCLAVLCG

SEQ ID NO: 48 - protein, 245 aa, Homo sapiens
SKEWQPAVQILLYSLIFLLSVLGNTLVITVLIRNKRTVTNIFLLSLAVSDLMLCLFCMPFNLIPNLLKFGSAVCKTTTYFMGTSVSVSTFNLVAISLERYGAICKQTKSHALKVIAATWCLSFTIMTPYPIYVMQQSWHTFLLLILFLIPGIVMMVAYGLISLEKKRVIRMLIVIVVLFFLCWMPIFSANAWRAYDTASAEPISFILLLSYTSSCVNPIIYCFMNKRFRLGFMATFPCCPNPGPP

SEQ ID NO: 49 - protein, 246 aa, Homo sapiens
SLNFDRAFLPALYSLLFLLGLLGNGAVAAVLLSRRLSSTDTFLLHLAVADTLLVLTLPLWAVDAAVQWVFGSGLCKVAGALFNINFYAGALLLACISFDRYLNIVHGPPARVTLTCLAVWGLCLLFALPDFIFVGRTALRVLQLVAGFLLPLLVMAYCYAHILAVRLRAMRLVVVVVVAFALCWTPYHLVVLVDILMDLGALAKSVTSGLGYMHCCLNPLLYAFVGVKFRERMWMLLLRLGCPNQR

SEQ ID NO: 50 - protein, 245 aa, Homo sapiens
NANFNKIFLPTIYSIIFLTGIVGNGLVILVMGYQKRSMTDKYRLHLSVADLLFVITLPFWAVDAVANWYFGNFLCKAVHVIYTVNLYSSVLILAFISLDRYLAIVHRKLLAEKVVYVGVWIPALLLTIPDFIFLWVVVFQFQHIMVGLILPGIVILSCYCIIISKKRKALKTTVILILAFFACWLPYYIGISIDSFILLEIIWISITEALAFFHCCLNPILYAFLGASRGSSLKILSKGKRGGHS

SEQ ID NO: 51 - protein, 246 aa, Homo sapiens
MASFKAVFVPVAYSLIFLLGVIGNVLVLVILERHRRSSTETFLFHLAVADLLLVFILPFAVAEGSVGWVLGTFLCKTVIALHKVNFYCSSLLLACIAVDRYLAIVHRRLLSIHITCGTIWLVGFLLALPEILFHAWFTSRFLYHVAGFLLPMLVMGWCYVGVVHRRQKAVRVAILVTSIFFLCWSPYHIVIFLDTLARLKAVAITMCEFLGLAHCCLNPMLYTFAGVKFRSDLSRLLTKLGCTGPA

SEQ ID NO: 52 - protein, 245 aa, Homo sapiens
ERAFGAQLLPPLYSLVFVIGLVGNILVVLVLVQYKKNMTSIYLLNLAISDLLFLFTLPFWIDYKLKDDFGDAMCKILSGFYYTGLYSEIFFIILLTIDRYLAIVHRTVTFGVITSIIIWALAILASMPGLYFEWKLFQALKLNLFGLVLPLLVMIICYTGIIKIKSKAVRLIFVIMIIFFLFWTPYNLTILISVFQDFLFTAVQVTEVIAYTHCCVNPVIYAFVGERFRKYLRQLFHRRVAVHLV

SEQ ID NO: 53 - protein, 246 aa, Homo sapiens
VKQIGAQLLPPLYSLVFIFGFVGNMLVVLILINCKKCLTDIYLLNLAISDLLFLITLPLWAHSAANEWVFGNAMCKLFTGLYHIGYFGGIFFIILLTIDRYLAIVHRTVTFGVVTSVITWLVAVFASVPGIIFGWNNFHTIMRNILGLVLPLLIMVICYSGILKTRHRAVRVIFTIMIVYFLFWTPYNIVILLNTFQEFFGLATQVTETLGMTHCCINPIIYAFVGEKFRSLFHIALGCRIAPLQK

SEQ ID NO: 54 - protein, 245 aa, Homo sapiens
TRALMAQFVPPLYSLVFTVGLLGNVVVVMILIKYRRIMTNIYLLNLAISDLLFLVTLPFWIHYVRGHNFGHGMCKLLSGFYHTGLYSEIFFIILLTIDRYLAIVHRTVTFGVITSIVTWGLAVLAALPEFIFSWRHFHTLRMTIFCLVLPLLVMAICYTGIIKTKYKAIRLIFVIMAVFFIFWTPYNVAILLSSYQSILFGVMLVTEVIAYSHCCMNPVIYAFVGERFRKYLRHFFHRHLLMHLG

SEQ ID NO: 55 - protein, 246 aa, Homo sapiens
IKAFGELFLPPLYSLVFVFGLLGNSVVVLVLFKYKRSMTDVYLLNLAISDLLFVFSLPFWGYYAADQWVFGLGLCKMISWMYLVGFYSGIFFVMLMSIDRYLAIVHRTLTYGVITSLATWSVAVFASLPGFLFTWKVLSSLEINILGLVIPLGIMLFCYSMIIRTKNKAVKMIFAVVVLFLGFWTPYNIVLFLETLVELEVLAIQATETLAFVHCCLNPIIYFFLGEKFRKYILQLFKTCRGLFVL

SEQ ID NO: 56 - protein, 245 aa, Homo sapiens
VKQIAARLLPPLYSLVFIFGFVGNMLVILILINCKKSMTDIYLLNLAISDLFFLLTVPFWAHYAAAQWDFGNTMCQLLTGLYFIGFFSGIFFIILLTIDRYLAVVHRTVTFGVVTSVITWVVAVFASLPGIIFFWKNFQTLKIVILGLVLPLLVMVICYSGILKTRHRAVRLIFTIMIVYFLFWAPYNIVLLLNTFQEFFGLAMQVTETLGMTHCCINPIIYAFVGEKFRNYLLVFFQKHIAKRF

SEQ ID NO: 57 - protein, 245 aa, Homo sapiens
VRQFSRLFVPIAYSLICVFGLLGNILVVITFAFYKRSMTDVYLLNMAIADILFVLTLPFWAVSHATGAFSNATCKLLKGIYAINFNCGMLLLTCISMDRYIAIVQRTLPRSKIICLVVWGLSVIISSSTFVFRWKLLMLGLELLFGFFIPLMFMIFCYTFIVKTRHKAIRVIIAVVLVFLACQIPHNMVLLVTAANLGKMNTKTVTEVLAFLHCCLNPVLYAFIGQKFRNYFLKILKDLWCVRRK

SEQ ID NO: 58 - protein, 246 aa, Homo sapiens
VRNFKAWFLPIMYSIICFVGLLGNGLVVLTYIYFKKTMTDTYLLNLAVADILFLLTLPFWAYSAAKSWVFGVHFCKLIFAIYKMSFFSGMLLLLCISIDRYVAIVQRVLLISKLSCVGIWILATVLSIPELLYEAFITIQVAQMVIGFLVPLLAMSFCYLVIIRTRNKAIKVIIAVVVVFIVFQLPYNGVVLAQTVANFNITAYDVTYSLACVRCCVNPFLYAFIGVKFRNDLFKLFKDLGCLSQE

SEQ ID NO: 59 - protein, 245 aa, Homo sapiens
IQTNGKLLLAVFYCLLFVFSLLGNSLVILVLVVCKRSITDVYLLNLALSDLLFVFSFPFQTYYLLDQWVFGTVMCKVVSGFYYIGFYSSMFFITLMSVDRYLAVVHRTIRMGTTLCLAVWLTAIMATIPLLVFKWKIFTNFKMNILGLLIPFTIFMFCYIKILHQKTKAIRLVLIVVIASLLFWVPFNVVLFLTSLHSMHILATHVTEIISFTHCCVNPVIYAFVGEPRESCEKSSSCQQHSSRS

SEQ ID NO: 60 - protein, 246 aa, Homo sapiens
VRQFASHFLPPLYWLVFIVGALGNSLVILVYWYCTKTMTDMFLLNLAIADLLFLVTLPFWAIAAADQWKFQTFMCKVVNSMYKMNFYSCVLLIMCISVDRYIAIAQKRLLYSKMVCFTIWVLAAALCIPEILYKLKSAVLTLKVILGFFLPFVVMACCYTIIIHTKHKALKVTITVLTVFVLSQFPYNCILLVQTIDAYAMFCFQVTQTIAFFHSCLNPVLYVFVGERFRRDLVKTLKNLGCISQA

SEQ ID NO: 61 - protein, 246 aa, Homo sapiens
VQAFSRAFQPSVSLTVAALGLAGNGLVLATHLAARRSPTSAHLLQLALADLLLALTLPFAAAGALQGWSLGSATCRTISGLYSASFHAGFLFLACISADRYVAIARSTPGRAHLVSVIVWLLSLLLALPALLFTVKGASAVAQVALGFALPLGVMVACYALLGRTRRRALRVVVALVAAFVVLQLPYSLALLLDTADLLAARALLVTSGLALARCGLNPVLYAFLGLRFRQDLRRLLRGGSSPSGP

SEQ ID NO: 62 - protein, 238 aa, Homo sapiens
ADRPHYNYYATLLTLLIAVIVFGNVLVCMAVSREKQTTTNYLIVSLAVADLLVATLVMPWVVYLEVVGFSRIHCDIFVTLDVMMCTASILNLCAISIDRYTAVAMSSKRRVTVMISIVWVLSFTISCPLLFGIANPAFVVYSSIVSFYVPFIVTLLVYIKIYIVEKKATQMLAIVLGVFIICWLPFFITHILNIHCDCNIPLYSAFTWLGYVNSAVNPIIYTTFNIEFRKAFLKILHC

SEQ ID NO: 63 - protein, 238 aa, Homo sapiens
SQARPHAYYALSYCALILAIVFGNGLVCMAVLKERQTTTNYLVVSLAVADLLVATLVMPWVVYLEVTGFSRICCDVFVTLDVMMCTASILNLCAISIDRYTAVVMSSCRRVALMITAVWVLAFAVSCPLLFGISNPDFVIYSSVVSFYLPFGVTVLVYARIYVVEKKATQMVAIVLGAFIVCWLPFFLTHVLNTHCQTCHVLYSATTWLGYVNSALNPVIYTTFNIEFRKAFLKILSC

SEQ ID NO: 64 - protein, 240 aa, Homo sapiens
GLAGQGAAALVGGVLLIGAVLAGNSLVCVSVATERQTPTNSFIVSLAAADLLLALLVLPLFVYSEVQGLSPRLCDALMAMDVMLCTASIFNLCAISVDRFVAVAVGGSRRQLLLIGATWLLSAAVAAPVLCGLEDRDYVVYSSVCSFFLPCPLMLLLYWATFRGERKAMRVLPVVVGAFLLCWTPFFVVHITQALCPACSVLVSAVTWLGYVNSALNPVIYTVFNAEFRNVFRKALRACC

SEQ ID NO: 65 - protein, 244 aa, Homo sapiens
RDFSVRILTACFLSLLILSTLLGNTLVCAAVIRFRSKVTNFFVISLAVSDLLVAVLVMPWKAVAEIAGPFGSFCNIWVAFDIMCSTASILNLCVISVDRYWAISSMTPKAAFILISVAWTLSVLISFIPVQLSLSRTYAISSSVISFYIPVAIMIVTYTRIYRIETKVLKTLSVIMGVFVCCWLPFFILNCILPFCGSGETTFDVFVWFGWANSSLNPIIYAFNADFRKAFSTLLGCYRLCPAT

SEQ ID NO: 66 - protein, 244 aa, Homo sapiens
PLGPSQVVTACLLTLLIIWTLLGNVLVCAAIVRSRANMTNVFIVSLAVSDLFVALLVMPWKAVAEVAGPFGAFCDVWVAFDIMCSTASILNLCVISVDRYWAISRMTQRMALVMVGLAWTLSILISFIPVQLSLNRTYAISSSLISFYIPVAIMIVTYTRIYRIETKVLKTLSVIMGVFVCCWLPFFILNCMVPFCSGHPETFDVFVWFGWANSSLNPVIYAFNADFQKVFAQLLGCSHFCSRT

SEQ ID NO: 67 - protein, 240 aa, Homo sapiens
MASPQLMPLVVVLSTICLVTVGLNLLVLYAVRSERHTVGNLYIVSLSVADLIVGAVVMPMNILYLLMSLGRPLCLFWLSMDYVASTASIFSVFILCIDRYRSVQQRTKTRASATILGAWFLSFLWVIPILGWYDVTWFKVMTAIINFYLPTLLMLWFYAKIYKAERKAAKQLGFIMAAFILCWIPYFIFFMVIAFCKNCCNLHMFTIWLGYINSTLNPLIYPLCNENFKKTFKRILHIRS

SEQ ID NO: 68 - protein, 245 aa, Homo sapiens
DSTACKITITVVLAVLILITVAGNVVVCLAVGLNRRNLTNCFIVSLAITDLLLGLLVLPFSAIYQLSCFGKVFCNIYTSLDVMLCTASILNLFMISLDRYCAVMDVTPVRVAISLVLIWVISITLSFLSIHLQVNEVYGLVDGLVTFYLPLLIMCITYYRIFKVEHKATVTLAAVMGAFIICWFPYFTAFVYRGLRGDDAILEAIVLWLGYANSALNPILYAALNRDFRTGYQQLFCCRLANRNS

SEQ ID NO: 69 - protein, 246 aa, Homo sapiens
TETLNKYVVIIAYALVFLLSLLGNSLVMLVILYSRRSVTDVYLLNLALADLLFALTLPIWAASKVNGWIFGTFLCKVVSLLKEVNFYSGILLLACISVDRYLAIVHQKRHLVKFVCLGCWGLSMNLSLPFFLFKWRMVLRILPHTFGFIVPLFVMLFCYGFTLRTKHRAMRVIFAVVLIFLLCWLPYNLVLLADTLMRTQVIALDATEILGFLHSCLNPIIYAFIGQNFRHGFLKILAMHGLVSKE

SEQ ID NO: 70 - protein, 246 aa, Homo sapiens
SLEINKYFVVIIYALVFLLSLLGNSLVMLVILYSRRSVTDVYLLNLALADLLFALTLPIWAASKVNGWIFGTFLCKVVSLLKEVNFYSGILLLACISVDRYLAIVHQKRYLVKFICLSIWGLSLLLALPVLLFNWRMLLRILPQSFGFIVPLLIMLFCYGFTLRTKHRAMRVIFAVVLIFLLCWLPYNLVLLADTLMRTQVIALDATEILGILHSCLNPLIYAFIGQKFRHGLLKILAIHGLISKD

SEQ ID NO: 71 - protein, 242 aa, Homo sapiens
AFCEQVFIKPEIFLSLGIVSLLENILVILAVVRNGHSPMYFFLCSLAVADMLVSVSNALETIMIAIVHFIQHMDNIFDSMICISLVASICNLLAIAVDRYVTIFYMTVRKALTLIVAIWVCCGVCGVVFIVYVHMFLFARLHVKRIAALPPADGVAPQQHSCMKGAVTITILLGVFIFCWAPFFLHLVLIITCPTNPYHFNTYLVLIMCNSVIDPLIYAFRSLELRNTFREILCGCNGMNLG

SEQ ID NO: 72 - protein, 239 aa, Homo sapiens
GCYEQLFVSPEVFVTLGVISLLENILVIVAIAKNKHSPMYFFICSLAVADMLVSVSNGSETIIITLLNFTVNIDNVIDSVICSSLLASICSLLSIAVDRYFTIFYMTVKRVGIIISCIWAACTVSGILFIIYVHMFLMARLHIKRIAVLPGTGAIRQGANMKGAITLTILIGVFVVCWAPFFLHLIFYISCPQNPYHFNLYLILIMCNSIIDPLIYALRSQELRKTFKEIICCYPLGGL

SEQ ID NO: 73 - protein, 240 aa, Homo sapiens
SPCEDMGIAVEVFLTLGVISLLENILVIGAIVKNKHSPMYFFVCSLAVADMLVSMSSAWETITIYLLNFVRHIDNVFDSMICISVVASMCSLLAIAVDRYVTIFYMTARRSGAIIAGIWAFCTGCGIVFILYIHMFLLARTHVKRIAALPGASSARQRTSMQGAVTVTMLLGVFTVCWAPFFLHLTLMLSCPQNLYHFNMYLILIMCNSVMDPLIYAFRSQEMRKTFKEIICCRGFRIAC

SEQ ID NO: 74 - protein, 245 aa, Homo sapiens
MPAWQIVLWAAAYTVIVVTSVVGNVVVMWIILAHKRTVTNYFLVNLAFAEASMAAFNTVVNFTYAVHNYGLFYCKFHNFFPIAAVFASIYSMTAVAFDRYMAIIHLSATATKVVICVIWVLALLLAFPQGYYIYEKVYHICVTVLIYFLPLLVIGYAYTVVGITKRKVVKMMIVVVCTFAICWLPFHIFFLLPYINPDLYLVYLAIMWLAMSSTMYNPIIYCCLNDRFRLGFKHAFRCCPFISAG

SEQ ID NO: 75 - protein, 245 aa, Homo sapiens
MPSWQLALWAPAYLALVLVAVTGNAIVIWIILAHRRTVTNYFIVNLALADLCMAAFNAAFNFVYASHNFGRAFCYFQNLFPITAMFVSIYSMTAIAADRYMAIVHLSAPSTKAVIAGIWLVALALASPQCFYKTLLLYHLVVIALIYFLPLAVMFVAYSVIGLTKKKFVKTMVLVVLTFAICWLPYHLYFILGSFQEDIYCVYLALFWLAMSSTMYNPIIYCCLNHRFRSGFRLAFRCCPWVTPT

SEQ ID NO: 76 - protein, 245 aa, Homo sapiens
MPSWRIALWSLAYGVVVAVAVLGNLIVIWIILAHKRTVTNYFLVNLAFSDASMAAFNTLVNFIYALHSFGANYCRFQNFFPITAVFASIYSMTAIAVDRYMAIIDLSATATKIVIGSIWILAFLLAFPQCLYKQHFTYHIIVIILVYCFPLLIMGITYTIVGITKRKVVKMMIIVVMTFAICWLPYHIYFILTAIYQQLNRVYLASFWLAMSSTMYNPIIYCCLNKRFRAGFKRAFRWCPFIKVS

SEQ ID NO: 77 - protein, 245 aa, Homo sapiens
MPPWAVALWSLAYGAVVAVAVLGNLVVIWIVLAHKRTVTNSFLVNLAFADAAMAALNALVNFIYALHGFGANYCRFQNFFPITAVFASIYSMTAIAVDRYMAIIDLSATATRIVIGSIWILAFLLAFPQCLYRQHFTYHMIVIVLVYCFPLLIMGITYTIVGITKRKVVKMMIIVVVTFAICWLPYHIYFILTAIYQQLNRVYLASFWLAMSSTMYNPIIYCCLNKRFRAGFKRAFRWCPFIHVS

SEQ ID NO: 78 - protein, 244 aa, Homo sapiens
LPLAMIFTLALAYGAVIILGVSGNLALIIIILKQKRNVTNILIVNLSFSDLLVAIMCLPFTFVYTLMDFGEAMCKLNPFVQCVSITVSIFSLVLIAVERHQLIINPNNRHAYVGIAVIWVLAVASSLPFLIYSHRLSYTTLLLVLQYFGPLCFIFICYFKIYIRTKRINIMLLSIVVAFAVCWLPLTIFNTVFDWNHQIIALFLLCHLTAMISTCVNPIFYGFLNKFRSRDDDYETIAMSTMHT

SEQ ID NO: 79 - protein, 245 aa, Homo sapiens
MLIEVQVVLILAYCSIILLGVIGNSLVIHVVIKFKRTVTNFFIANLAVADLLVNTLCLPFTLTYTLMGMGPVLCHLVPYAQGLAVQVSTITLTVIALDRHRCIVYISKRISFLIIGLAWGISALLASPLAIFIYGTVYSLSSLLILYVLPLGIISFSYTRIWSKRQKTTKMLVCVVVVFAVSWLPLHAFQLAVDIDSQVLDIFTVFHIIAMCSTFANPLLYGWMNSNYRKAFLSAFRCEQRLDAI

SEQ ID NO: 80 - protein, 245 aa, Homo sapiens
DSVDVMVFIVTSYSIETVVGVLGNLCLMCVTVRQKANVTNLLIANLAFSDFLMCLLCQPLTAVYTIMDFGETLCKMSAFIQCMSVTVSILSLVLVALERHQLIINPSISQAYLGIVLIWVIACVLSLPFLANHHRTIYTTFLLLFQYCLPLGFILVCYARIYRRMKQVNVVLVVMVVAFAVLWLPLHVFNSLEDWHHEAIPIFLVCHLLAMASTCVNPFIYGFLNTNFKKEIKALVLTCQQSAPL

SEQ ID NO: 81 - protein, 241 aa, Homo sapiens
SVDDLQYFLIGLYTFVSLLGFMGNLLILMALMKKRKTTVNFLIGNLAFSDILVVLFCSPFTLTSVLLDFGKVMCHIMPFLQCVSVLVSTLILISIAIVRYHMIKHLTANHGYFLIATVWTLGFAICSPLPVFSYRIAFTISLLLVQYILPLVCLTVSHTSVCRSSRSVFYRLTILILVFAVSWMPLHLFHVVTDFNDNLISVYCICHLLGMMSCCLNPILYGFLNNGIKADLVSLIHCLHM

SEQ ID NO: 82 - protein, 244 aa, Homo sapiens
SSLALAIAITALYSAVCAVGLLGNVLVMFGIVRYTKTATNIYIFNLALADALATSTLPFQSAKYLMETFGELLCKAVLSIDYYNMFTSIFTLTMMSVDRYIAVCHRTPAKAKLINICIWVLASGVGVPIMVMYWDTVTKICVFLFAFVVPILIITVCYGLMLLRLRRITRMVLVVVGAFVVCWAPIHIFVIVWTLVDIDRRALHLCIALGYANSSLNPVLYAFLDEFRQLCRKPCGRPDPSSFS

SEQ ID NO: 83 - protein, 244 aa, Homo sapiens
ISPAIPVIITAVYSVVFVVGLVGNSLVMFVIIRYTKTATNIYIFNLALADALVTTTMPFQSTVYLMNSFGDVLCKIVISIDYYNMFTSIFTLTMMSVDRYIAVCHRTPLKAKIINICIWLLSSSVGISAIVLWWDLFMKICVFIFAFVIPVLIIIVCYTLMILRLRRITRLVLVVVAVFVVCWTPIHIFILVEALGSTSHSSYYFCIALGYTNSSLNPILYAFLDEFRDFCFPLKMRMERQSTS

SEQ ID NO: 84 - protein, 244 aa, Homo sapiens
PSMITAITIMALYSIVCVVGLFGNFLVMYVIVRYTKTATNIYIFNLALADALATSTLPFQSVNYLMGTFGTILCKIVISIDYYNMFTSIFTLCTMSVDRYIAVCHRTPRNAKIINVCNWILSSAIGLPVMFMYWENLLKICVFIFAFIMPVLIITVCYGLMILRLRRITRMVLVVVAVFIVCWTPIHIYVIIKALVTIPETSWHFCIALGYTNSCLNPVLYAFLDEFREFCIPTSSNIEQQNST

SEQ ID NO: 85 - protein, 244 aa, Homo sapiens
LPLGLKVTIVGLYLAVCVGGLLGNCLVMYVILRHTKTATNIYIFNLALADTLVLLTLPFQGTDILLGFFGNALCKTVIAIDYYNMFTSTFTLTAMSVDRYVAICHRTSSKAQAVNVAIWALASVVGVPVAIMYWGPVFAICIFLFSFIVPVLVISVCYSLMIRRLRRITRLVLVVVAVFVGCWTPVQVFVLAQGLGVQPSSILRFCTALGYVNSCLNPILYAFLDEFRKFCCASALRRDVQVSD

SEQ ID NO: 86 - protein, 244 aa, Homo sapiens
APVWAFYLQAAFMGTVFLIGFPLNAMVLVATLRYKKLRQPLNYILVNVSFGGFLLCIFSVFPVFVASFGRHVCALEGFLGTVAGLVTGWSLAFLAFERYIVICKFSSKHALTVVLATWTIGIGVSIPPFFGYRSESYTWFLFIFCFIVPLSLICFSYTQLLRAEREVSRMVVVMVGSFCVCYVPYAAFAMYMVNNRNHGLLVTIPSFFSKSACIYNPIIYCFMNKQFQACIMKMVCGKAMTDES

SEQ ID NO: 87 - protein, 245 aa, Homo sapiens
AEPWQFSMLAAYMFLLIVLGFPINFLTLYVTVQHKRTPLNYILLNLAVADLFMVLGGFTSTLYTSLHGFGPTGCNLEGFFATLGGEIALWSLVVLAIERYVVVCKFGENHAIMGVAFTWVMALACAAPPLAGVNNESFVIYMFVVHFTIPMIIIFFCYGQLVFTEKEVTRMVIIMVIAFLICWVPYASVAFYIFTHQGSNFFMTIPAFFAKSAAIYNPVIYIMMNKQFRNCMLTTICCGKNPLGD

SEQ ID NO: 88 - protein, 245 aa, Homo sapiens
APRWVYHLTSVWMIFVVIASVFTNGLVLAATMKFKRHPLNWILVNLAVADLAETVIASTISVVNQVYGLGHPMCVLEGYTVSLCGITGLWSLAIISWERWMVVCKFDAKLAIVGIAFSWIWAAVWTAPPIFGPGVQSYMIVLMVTCCITPLSIIVLCYLQVWLAEKEVTRMVVVMVLAFCFCWGPYAFFACFAAANPGYPFMAALPAFFAKSATIYNPVIYVFMNRQFRNCILQLFGKKVDDGSE

SEQ ID NO: 89 - protein, 245 aa, Homo sapiens
APRWVYHLTSVWMIFVVTASVFTNGLVLAATMKFKRHPLNWILVNLAVADLAETVIASTISIVNQVSGLGHPMCVLEGYTVSLCGITGLWSLAIISWERWLVVCKFDAKLAIVGIAFSWIWSAVWTAPPIFGPGVQSYMIVLMVTCCIIPLAIIMLCYLQVWLAEKEVTRMVVVMIFAYCVCWGPYTFFACFAAANPGYAFMAALPAYFAKSATIYNPVIYVFMNRQFRNCILQLFGKKVDDGSE

SEQ ID NO: 90 - protein, 245 aa, Homo sapiens
MSQTEHNIVATYLIMAGMISIISNIIVLGIFIKYKRTPTNAIIINLAVTDIGVSSIGYPMSAASDLYGFGYAGCQVYAGLNIFFGMASIGLLTVVAVDRYLTICLMTTNTYIGLILGAWINGLFWALMPIIGRSFVSYTMTVIAINFIVPLTVMFYCYYHVTLSQIDVTKMSVIMICMFLVAWSPYSIVCLWASFGDPKKIMAIIAPLFAKSSTFYNPCIYVVANKKFRRAMLAMFKCQTHQTMP

SEQ ID NO: 91 - protein, 240 aa, Homo sapiens
EGQGSAILISFIYSVVCLVGLCGNSMVIYVILRYAKTATNIYILNLAIADELLMLSVPFLVTSTLLRHFGALLCRLVLSVDAVNMFTSIYCLTVLSVDRYVAVVHRRPTVAKVVNLGVWVLSLLVILPIVVFRWLVGFVLYTFLMGFLLPVGAICLCYVLIIAKERKITLMVMMVVMVFVICWMPFYVVQLVNVFAEQDDATVSQLSVILGYANSCANPILYGFLSDFRNGTCTSRITTL

SEQ ID NO: 92 - protein, 245 aa, Homo sapiens
YDLTSNAVLTFIYFVVCIIGLCGNTLVIYVILRYAKTITNIYILNLAIADELFMLGLPFLAMQVALVHFGKAICRVVMTVDGINQFTSIFCLTVMSIDRYLAVVHRRPRTAKMITMAVWGVSLLVILPIMIYAWYTGFIIYTFILGFLVPLTIICLCYLFIIIKEKKVTRMVSIVVAVFIFCWLPFYIFNVSSVSMAISPTMFDFVVVLTYANSCANPILYAFLSDNFKKSFQNVLCLVKVSGTD

SEQ ID NO: 93 - protein, 244 aa, Homo sapiens
GLAVSGVLIPLVYLVVCVVGLLGNSLVIYVVLRHTPSVTNVYILNLALADELFMLGLPFLAAQNALSYFGSLMCRLVMAVDGINQFTSIFCLTVMSVDRYLAVVHRTAPVARTVSAAVWVASAVVVLPVVVFAWRAGFIIYTAALGFFGPLLVICLCYLLIVVKERRVTRMVVAVVALFVLCWMPFYVLNIVNVVCPLPEELYFLVVALPYANSCANPILYGFLSYFRRVLLRPSRRVRSQEPT

SEQ ID NO: 94 - protein, 246 aa, Homo sapiens
ARAAGMVAIQCIYALVCLVGLVGNALVIFVILRYAKTATNIYLLNLAVADELFMLSVPFVASSAALRHFGSVLCRAVLSVDGLNMFTSVFCLTVLSVDRYVAVVHRRPSVAKLINLGVWLASLLVTLPIAIFAWSAVFVVYTFLLGFLLPVLAIGLCYLLIVGKEKKITRLVLMVVVVFVLCWMPFYVVQLLNLVVTSLDATVNHVSLILSYANSCANPILYGFLSDNFRRSFQRVLCLRCCLLEG

SEQ ID NO: 95 - protein, 245 aa, Homo sapiens
SAGARAVLVPVLYLLVCAAGLGGNTLVIYVVLRFAKTVTNIYILNLAVADVLYMLGLPFLATQNAASFFGPVLCRLVMTLDGVNQFTSVFCLTVMSVDRYLAVVHRRPRVAKLASAAAWVLSLCMSLPLLVFLWGAVFIIYTAVLGFFAPLLVICLCYLLIVVKERKVTRMVLVVVLVFAGCWLPFFTVNIVNLAVALPQELYFFVVILSYANSCANPVLYGFLSDNFRQSFQKVLCLRKGSGAK

SEQ ID NO: 96 - protein, 245 aa, Homo sapiens
EDIMGYNILRVLIWFISILAITGNIIVLVILTTSQLTVPRFLMCNLAFADLCIGIYLLLIASVDIHTKQTGAGCDAAGFFTVFASELSVYTLTAITLERWHTITHVQLRHAASVMVMGWIFAFAAALFPIFGISSYMKVSICLPMDIDSPLSQLYVMSLLVLNVDTRIAKRMAMLIFTDFLCMAPISFFAISASLKVPLITAKILLVLFHPINSCANPFLYAIFTKNFRRDFFILLSKCGCYEMQ

SEQ ID NO: 97 - protein, 238 aa, Homo sapiens
EDIMGYDFLRVLIWLINILAIMGNMTVLFVLLTSRLTVPRFLMCNLSFADFCMGLYLLLIASVDSQTKQTGSGCSTAGFFTVFASELSVYTLTVITLERWHTITYLRLRHAILIMLGGWLFSSLIAMLPLVGVSNYMKVSICFPMDVETTLSQVYILTDTKIAKKMAILIFTDFTCMAPISFFAISAAFKVPLITSKVLLVLFYPINSCANPFLYAIFTKQRDFFLLLSKFGCCKRRA

SEQ ID NO: 98 - protein, 239 aa, Homo sapiens
ARCLEVSISDGLFLSLGLVSLVENALVVATIAKNRHSPMYCFICCLALSDLLVSGTNVLETAVILLLEVLQQLDNVIDVITCSSMLSSLCFLGAIAVDRYISIFYVTLPRAPRAVAAIWVASVVFSTLFIAYYDHVAVLLCLVVFFLAMLVLMAVLYVHMLARGLKGAVTLTILLGIFFLCWGPFFLHLTLIVLCPEHPTNFNLFLALIICNAIIDPLIYAFHSQELRRTLKEVLTCSW